(19) United States
(12) Patent Application Publication
(10) Pub. No.: US 2017/0247715 A1
(43) Pub. Date: Aug. 31, 2017

(54) GENETICALLY MODIFIED PLANTS HAVING ALTERED LIGNIN CONTENT

(71) Applicants: SweTree Technologies AB, Umeå (SE); VIB VZW, Gent (BE); Universiteit Gent, Gent (BE)

(72) Inventors: Wout BOERJAN, Kalken (BE); Ruben VANHOLME, Destelbergen (BE); Hannele TUOMINEN, Umeå (SE); Halbay TURUMTAY, Gent (BE); Sacha ESCAMEZ, Umeå (SE); Bo ZHANG, Umeå (SE)

(73) Assignees: SweTree Technologies AB, Umeå (SE); VIB VZW, Gent (BE); Universiteit Gent, Gent (BE)

(21) Appl. No.: 15/504,288

(22) PCT Filed: Aug. 20, 2015
(86) PCT No.: PCT/SE2015/050884

§ 371 Date: Feb. 15, 2017

(30) Foreign Application Priority Data
Aug. 20, 2014 (SE) 1450967-3

(51) Int. Cl. C12N 15/82; C07K 14/415

(52) U.S. Cl. CPC C12N 15/8255; C07K 14/415

(57) ABSTRACT

The invention relates to methods for producing a plant with altered lignin content by genetically manipulating expression of a PIRIN gene. The said method comprises (a) altering expression of a gene encoding a PIRIN2 polypeptide in the plant; and/or (b) altering expression of a gene encoding a PIRIN4 polypeptide in the plant. A plant with reduced lignin content is produced by increasing expression of a gene encoding a PIRIN2 polypeptide in the plant or decreasing expression of a gene encoding a PIRIN4 polypeptide in the plant. The invention further relates to genetically modified plants produced by the said methods.

### TECHNICAL FIELD

The invention relates to methods for producing a plant with altered lignin content by genetically manipulating expression of a PIRIN gene. The said method comprises (a) altering expression of a gene encoding a PIRIN2 polypeptide in the plant; and/or (b) altering expression of a gene encoding a PIRIN4 polypeptide in the plant. The invention further relates to genetically modified plants produced by the said methods.

### BACKGROUND ART

Lignin is one the main constituents of the woody biomass of forest trees. It is crucial in the tree trunk since it confers physical strength to the stem as well as resistance against wood-decaying pathogens. However, lignin does not contribute to the yield in either kraft pulping or during saccharification (hydrolysis of the woody raw material into its constituent sugar moieties), and causes in fact serious problems in both processes.

During kraft pulping, lignin has to be removed in expensive and environmentally risky processes since it otherwise impairs the quality of the end products. During saccharification, lignin makes access of hydrolytic enzymes to cellulose difficult and in thermochemical pretreatment steps partial degradation of lignin causes formation of phenolic fermentation inhibitors that reduce the productivity. These problems related to lignin have raised a vast interest to both understand the chemical and molecular basis of lignin homeostasis in forest trees and to identify ways to decrease the negative effects of lignin by molecular breeding techniques (see for instance Gomez et al., 2008; Halpin, 2004, Li et al., 2008). Two main approaches have been taken, and these are described shortly below.

The first approach concerns reduction of the content of lignin in the tree trunk. Expression of several genes encoding proteins of the lignin biosynthetic pathway has been modified to reduce the total amount of lignin (Boerjan et al, 2003; Chiang, 2006). Several of these studies have demonstrated that lignin is the main determinant of both the pulping efficiency and the saccharification potential (sugar yield); the more lignin the lignocellulosic raw material contains, the more difficult it is to hydrolyse lignocellulose polysaccharides by using chemicals or enzymes (Baucher et al., 2003). However, it has also become clear that a reduction in the content of lignin is often accompanied by a decrease in the growth of the plants, and this approach does therefore not seem to be the right choice to improve the sustainable utilization of the lignocellulose (reviewed in Li et al., 2008).

An alternative approach instead involves modification of lignin composition. Lignin is a polymer of three different monolignols; coniferyl alcohol, sinapyl alcohol, and p-coumaryl alcohol, which, when polymerised, form guaiacyl (G), syringyl (S) and p-hydroxyphenyl (H) lignin units, respectively. The monomeric composition of lignin, and especially the ratio between S and G units of lignin, has a great impact on the processing properties of the wood. Higher proportion of the S type of lignin is generally preferred since it makes lignin more easily hydrolysable and has been seen to improve pulping performance (Pilate et al., 2002). High ratio between S and G type lignin seems to promote also the saccharification yield (Studer et al., 2011) even though this relationship is not always clear (Chen and Dixon 2007; Dien et al., 2011). Even though the H units normally constitute only a minor portion of the total lignin (except for in grasses), they are also of interest since they may affect the energy content. In conclusion, it is important that we can modify lignin composition in different ways in order to facilitate different end uses of lignocellulose. Today, we still have only limited knowledge of the different ways to affect the lignin composition, and it is widely recognized that additional tools are needed.

Pirin proteins are members of an iron-containing subgroup of the cupin superfamily. In Arabidopsis thaliana, four proteins have been identified in the Pirin family (see e.g. the UniProt database; www.uniprot.org): Pirin-1 (At3g59220); Pirin-like protein At2g43120 (hereinafter referred to as PIRIN2); Pirin-like protein At1g50590 (hereinafter referred to as PIRIN4); and Putative pirin-like protein At3g59260. Amino acid alignment shows that there is a 75% identity between A. thaliana PIRIN1 (SEQ ID NO: 2) and PIRIN2 (SEQ ID NO: 4). There is a 55% identity between PIRIN1 and PIRIN4 (SEQ ID NO: 6). A corresponding nucleotide sequence alignment shows a 79% identity between the PIRIN1 (SEQ ID NO: 1) and PIRIN2 (SEQ ID NO: 3) genes and a 61% identity between the PIRIN1 and PIRIN4 (SEQ ID NO: 5) genes.

Proteins in the Pirin family have also been identified in other plant species such as Populus trichocarpa (SEQ ID NO: 7 to 10), Eucalyptus grandis (SEQ ID NO: 11 to 16) and corn Zea mays (SEQ ID NO: 17 to 28). Other proteins from the Pirin family are found in the Phytozome database (www.phytozome.org).

WO 2012/027609 discloses methods of making and using plant extracts that include quercetin, which are generated from transgenic plants that have modulated Pirin-1 activity. There is no mention of modified plants having modulated lignification properties.

Vanholme, R. et al. (2012b) discloses a systems biology approach to study the plant's response to lignin perturbations. Inflorescence stems of 20 Arabidopsis thaliana mutants, each mutated in a single gene of the lignin biosynthetic pathway were analyzed by transcriptomics and metabolomics. Genes with a putative role in phenolic metabolism were identified.

Pesquet, E. et al. (2013) discloses screening for differentially expressed genes in Zinnia elegans cell culture system where vessel-like tracheary elements (TEs) differentiate in a semisynchronous manner after addition of a hormonal stimulus. Differentially expressed genes were identified in a condition where normal differentiation of TEs was blocked by addition of silver thiosulfate (STS).

The abstract “Modification of lignin composition for sustainable utilization of woody biomass” (retrieved from www.sciencenet.se/converis/publicweb/contract/14475) discloses a plant model system wherein a low activity of (unspecified) Pirin protein resulted in a low percentage of G lignin without changing the total amount of lignin. The abstract does not teach which Pirin genes that should be modulated in order to achieve a modulated lignin composition.

Yanofksy et al. (U.S. Pat. No. 6,410,826) provides methods of selectively controlling lignin biosynthesis in plants such that lignification is reduced or enhanced. The invention provides a method of reducing lignification in a vascular plant by ectopically expressing a nucleic acid molecule encoding an AGL8-like gene product in the plant, whereby lignification is reduced due to ectopic expression of the nucleic acid molecule.

Consequently, there is a need for identifying additional genes that specifically regulate the lignification process and for methods of genetically modifying plants to reduce their lignin content.

### BRIEF DESCRIPTION OF THE DRAWINGS

FIG. 1. The expression of PIRIN2 and PIRIN4 in a set of lignin mutants. Values are ²log(expression in mutant/expression in WT24 cm). PAL2, C4H, 4CL2 and C3H1 are given as representative genes involved in lignification. G(8-O-4)G, G(8-O-4)G(8-O-4)G and G(8-O-4)G′(8-O-4)G are given as representative G-type oligolignols.

FIG. 2. The expression of PIRIN2 (A) and PIRIN4 (B) in a set of lignin mutants as determined by RT-qPCR. Expression data for PIRINs is normalized to ACTIN (ACT) expression, hereafter the data was normalized to the sample with the highest PIRIN/ACT ratio, resulting in a value of 1 for wt_3 in panel A and for wt_2 in panel B.

FIG. 3. The expression of PIRIN2 and PIRIN4 in lignifying cell cultures. A) Genes involved in lignification (4CL1, PALI, CCoAOMTI, COMT) are strongly upregulated 24 hours after induction, thereafter they show a decrease. B) and C) PIRIN2 and 4 are upregulated 24 hours after induction (line, “lign”), thereafter they show a decrease. No upregulation was measured without induction (line, “unlig”).

FIG. 4. Expression of PIRIN2 and PIRIN4 in Arabidopsis TE cell cultures. Relative expression levels of PIRIN2 and PIRIN4 are shown in cell cultures 0, 1, 2, 3, 4, 5, 6, 7, 9, 10 days after induction of TE differentiation. Arabidopsis UBQ10 and SAND were used as reference genes.

FIG. 5. Targeted search for oligolignols in inflorescence stems (about 48 cm in height for A, and 28 cm for B). A significant increase in S-unit containing oligolignols was observed in pirin2 mutants, whereas a significant decrease was observed for this type of oligolignols in pirin4 mutants. Results normalized to their respective WT with the average in the first column and standard error in the second. Significant increases are indicated in bold values, significant decreases are underlined.

FIG. 6. Lignin amount as determined by the acetylbromide method. Error bars represent the standard errors. *0.01<p<0.05, **p<0.01

FIG. 7. Verification of the changes in lignin composition and biosynthetic activities in the pirin KO mutants and OE lines. (a-e) Maule staining of wild type (a), pirin2-1 (b), pirin2-2 (c), PIRIN2 OE6 (d) and PIRIN2 OE13 (e). (f,g) Py-GC/MS. Relative proportion of total lignin (f) and S/G ratios of lignin (g) in the secondary walls of 8-week-old hypocotyls. (h) Realtime qPCR of lignin biosynthetic genes. The asterisks indicate statistically significant difference from the wild type by t-test with Welch correction (** P<0.01). Vertical bars indicate ±SD.

FIG. 8. Saccharification analysis. A) pirin2-2 without pretreatment B) pirin2-2 with acid pretreatment C) PIRIN2 OE6 without pretreatment and D) pirin4-4 without pretreatment.

### DISCLOSURE OF THE INVENTION

It has surprisingly been found that lignin amount is significantly decreased in Arabidopsis thaliana cell lines overexpressing PIRIN2, as well as in cell lines having reduced expression of PIRIN4. Further, such cell lines show increased saccharification yield and a decreased ratio between syringyl and guaiacyl lignin units (S/G ratio). In contrast, lowering expression of PIRIN2 results in increased lignin amounts, increased S/G ratio and reduced saccharification ability. It is concluded that lignification can be modulated by altering expression of PIRIN2 and/or PIRIN4 (but not PIRIN1) in Arabidopsis.

Consequently, in a first aspect the invention provides a method for producing a plant, such as a woody plant, with reduced lignin content by genetically manipulating expression of a PIRIN gene, said method comprising:

(a) increasing expression of a gene encoding a PIRIN2 polypeptide in the plant; and/or

(b) decreasing expression of a gene encoding a PIRIN4 polypeptide in the plant;

wherein the amino acid sequence of the PIRIN2 polypeptide has at least 60% sequence identity to SEQ ID NO: 4, and wherein the amino acid sequence of the PIRIN4 polypeptide has at least 60% sequence identity to SEQ ID NO: 6.

The term “reduced lignin content” means a significantly decreased extent of lignification in one or more tissues as compared to the extent of lignification in a corresponding wild type plant. The term “reduced” also encompasses lignification that is significantly decreased in one or more tissues while wild type levels of lignification persist elsewhere in the vascular plant.

The term “decreasing expression” is intended to encompass well known equivalent terms regarding expression such as “inhibiting”, “down-regulating”, “knocking out”, “silencing”, etc.

The term “increasing expression” is intended to encompass well known methods to increase the expression by regulatory sequences, such as promoters, or proteins, such as transcription factors.

The methods according to the invention are furthermore useful for producing plants having a decreased ratio between syringyl and guaiacyl lignin units (S/G ratio) and/or having increased saccharification potential. The term “saccharification” refers to the process by which the woody raw material is converted by hydrolysis of into its constituent sugar moieties.

As mentioned above, the invention provides a method comprising increasing expression of a gene encoding a PIRIN2 polypeptide in a plant. Preferably, the said plant is transformed with a transgene encoding the PIRIN2 polypeptide. The term “transgene” refers to a gene or genetic material that has been transferred by genetic engineering techniques into the plant cell. “Transgenic plants” or “transformed plants” refers to plants that have incorporated or integrated exogenous nucleic acid sequences or DNA fragments into the plant cell. These nucleic acid sequences include those that are exogenous, or not present in the untransformed plant cell, as well as those that may be endogenous, or present in the untransformed plant cell.

Preferably, the transgene comprises a homologous or heterologous promoter operably linked to a DNA molecule encoding the PIRIN2 polypeptide. For example, the promoter of an endogenous gene encoding the PIRIN2 polypeptide can be genetically manipulated to increase expression of the PIRIN2 polypeptide in the plant. Alternatively, the promoter can be a constitutive promoter, such as the CaMV 35S promoter. The term “constitutive promoter” means a promoter which induce the expression of the downstream-located coding region in all tissues irrespective of environmental or developmental factors. “Heterologous” generally refers to nucleic acid sequences that are not endogenous to the cell or part of the native genome in which they are present, and have been added to the cell by infection, transfection, microinjection, electroporation, microprojection, or the like.

By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

When the method involves increasing expression of a gene encoding a PIRIN2 polypeptide in the plant, the said PIRIN2 polypeptide is preferably selected from:

(a) a polypeptide consisting of the amino acid sequence shown as SEQ ID NO: 4;

(b) a polypeptide comprising the amino acid sequence shown as SEQ ID NO: 4;

(c) a polypeptide which is an ortholog in a plant species of the Arabidopsis thaliana PIRIN2 polypeptide,

which ortholog has at least 60% identity with the amino acid sequence shown as SEQ ID NO: 4; and which is capable of reducing lignin content in a plant.

The term “ortholog” means a member of a group of (orthologous) polypeptide or genes in different species, which have evolved from a common ancestral polypeptide/gene by speciation and which essentially retain the same biological function.

In a preferred aspect, the PIRIN2 polypeptide, including orthologs thereof, has at least 60% identity, such as at least 70%, 75%, 80%, 85%, 90%, or 95%, with the amino acid sequence shown as SEQ ID NO: 4. However, the invention encompasses polypeptides carrying modifications like substitutions, small deletions, insertions or inversions, which polypeptides nevertheless have substantially the biological activities of a PIRIN2 polypeptide.

In a corresponding manner, the said gene encoding a PIRIN2 polypeptide is selected from:

(a) a nucleic acid molecule consisting of the nucleotide sequence shown as SEQ ID NO: 3;

(b) a nucleic acid molecule comprising the nucleotide sequence shown as SEQ ID NO: 3;

(c) a nucleic acid molecule which is capable of hybridizing, under stringent hybridization conditions, to a nucleotide sequence complementary to SEQ ID NO: 3; and

(d) a nucleic acid molecule which is an ortholog in a plant species of the Arabidopsis thaliana PIRIN2 gene, which ortholog has at least 65% identity with the nucleotide sequence shown as SEQ ID NO: 3; and which encodes a polypeptide capable of reducing lignin content in a plant.

Preferably the said stringent hybridization conditions are moderate stringency hybridization conditions, and more preferably high stringency hybridization conditions. By “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Stringency can typically be increased by reducing the concentration of salt, increasing the concentration of formamide, or raising the hybridization temperature. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours.

In a preferred aspect, the said gene encoding a PIRIN2 polypeptide, including orthologs thereof, has at least 65% identity, such as at least 70%, 75%, 80%, 85%, 90%, or 95%, with the nucleotide sequence shown as SEQ ID NO: 3. However, the invention encompasses polynucleotides carrying modifications like substitutions, small deletions, insertions or inversions, which nevertheless encode polypeptides having substantially the biological activity of the PIRIN2 polypeptide. Included in the invention is also a polynucleotide which nucleotide sequence is degenerate, because of the genetic code, to the nucleotide sequence shown as SEQ ID NO: 3. Since there are 64 possible codons, but only 20 natural amino acids, most amino acids are coded for by more than one codon. This natural “degeneracy”, or “redundancy”, of the genetic code is well known in the art. It will thus be appreciated that SEQ ID NO: 3 is only an example within a large but definite group of sequences which will encode the PIRIN2 polypeptide.

As mentioned above, the invention provides a method comprising decreasing expression of a gene encoding a PIRIN4 polypeptide in a plant. Methods for decreasing expression of specific genes are known in the art and includes e.g. RNA interference and T-DNA insertion.

The term “RNA interference” (RNAi) refers to well-known methods for down-regulating or silencing expression of a naturally occurring gene in a host plant. RNAi employs a double-stranded RNA molecule or a short hairpin RNA to change the expression of a nucleic acid sequence with which they share substantial or total homology. For a review, see e.g. Agrawal, N. et al (2003) Microbiol Mol Biol Rev. 67(4): 657-685. Consequently, expression of the PIRIN4 gene can be down-regulated by introducing into at least one plant cell a nucleic acid molecule encoding a ribonucleic acid sequence, which is capable of forming a double-stranded ribonucleic acid molecule, whereby a fragment of said double-stranded ribonucleic acid molecule has a nucleic acid sequence having at least 50% nucleic acid sequence identity to the PIRIN4 gene.

The term “T-DNA insertion” refers to methods utilizing transfer-DNA (T-DNA) for disrupting genes via insertional mutagenesis. Down-regulating or silencing expression of the PIRIN4 gene in a plant cell can thus be achieved by T-DNA mutagenesis, wherein the T-DNA is used to randomly introduce mutations in the plant genome followed by selecting plants comprising silencing mutations in the endogenous PIRIN4 gene. The plant, or plant cell, in which the endogenous PIRIN4 gene is mutated can later be identified by PCR or other high-throughput technologies. For a review of T-DNA as an insertional mutagen, see e.g. Krysan, P. J. et al. (1999) Plant Cell, 11: 2283-2290.

Additional methods for reducing gene expression in plants are known in the art and are exemplified by the following non-limiting examples:
(a) The down-regulation or silencing of expression of a PIRIN4 nucleic acid molecule in a plant cell can be achieved by means of mutations, such as point mutations, in the PIRIN4 genes. Mutations can be introduced randomly into the genome of a plant cell, and mutagenized plant cells can be selected by specific methods such like TILLING (Targeting Induced Local Lesions IN Genomes). Down-regulation or silencing of expression of a PIRIN4 gene can also result from natural mutations occurring in natural plant populations, which result in (non-silent) silencing mutations in the PIRIN4 genes. EcoTILLING is a method that uses the TILLING method to identify these natural occurring mutations (polymorphisms) in plant populations.
(b) Vectors expressing an untranslatable form of a gene, e.g., sequences comprising one or more stop codons, or nonsense mutation, can be used to down-regulate or silence the expression of a PIRIN4 nucleic acid molecule in a plant cell. In particular, such constructs can be made by introducing a premature stop codon into the gene. One way of performing targeted DNA insertion is by use of the retrovirus DNA integration machinery, as described in WO 2006/078431.
(c) Down-regulating or silencing expression of a naturally occurring PIRIN4 gene in a host plant can be achieved by transforming into the host plant a transgene comprising an anti-sense sequence of a PIRIN4 nucleic acid molecule. The objective of the antisense approach is to use a sequence complementary to the target gene to block its expression and create a mutant cell line or organism in which the level of a single chosen protein is selectively reduced or abolished.

Additional methods might be selected from the resent years of development of methods and compositions to target and cleave genomic DNA by site specific nucleases e.g. Zinc Finger Nucleases, ZFNs, Meganucleases, Transcription Activator-Like Effector Nucelases, TALENS and Clustered Regularly Interspaced Short Palindromic Repeats/CRISPR-associated nuclease (CRISPR/Cas) with an engineered crRNA/tracr RNA), to induce targeted mutagenesis, induce targeted deletions of cellular DNA sequences, and facilitate targeted recombination of an exogenous donor DNA polynucleotide within a predetermined genomic locus. Current methods for targeted insertion of exogenous DNA typically involve co-transformation of plant tissue with a donor DNA polynucleotide containing at least one transgene and a site specific nuclease, e.g., ZFN, which is designed to bind and cleave a specific genomic locus of an actively transcribed coding sequence. This causes the donor DNA polynucleotide to stably insert within the cleaved genomic locus resulting in targeted gene addition at a specified genomic locus comprising an actively transcribed coding sequence.

As used herein the term “zinc fingers,” defines regions of amino acid sequence within a DNA binding protein binding domain whose structure is stabilized through coordination of a zinc ion.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. No. 6,453,242; see also WO 98/53058, each of which is herein incorporated by reference.

A “TALE DNA binding domain” or “TALE” is a polypeptide comprising one or more TALE repeat domains/units. The repeat domains are involved in binding of the TALE to its cognate target DNA sequence.

A single “repeat unit”, also referred to as a “repeat”, is typically 33-35 amino acids in length and exhibits at least some sequence homology with other TALE repeat sequences within a naturally occurring TALE protein. See, e.g., U.S. Patent Publication No. 2011/0301073, incorporated by reference herein in its entirety.

The CRISPR (Clustered Regularly Interspaced Short Palindromic Repeats)/Cas (CRISPR Associated) nuclease system. Briefly, a “CRISPR DNA binding domain” is a short stranded RNA molecule that acting in concert with the CAS enzyme can selectively recognize, bind, and cleave genomic DNA. The CRISPR/Cas system can be engineered to create a double-stranded break (DSB) at a desired target in a genome, and repair of the DSB can be influenced by the use of repair inhibitors to cause an increase in error prone repair. See, e.g., Jinek et al (2012) Science 337, p. 816-821, incorporated by reference herein in its entirety.

Zinc finger, CRISPR and TALE binding domains can be “engineered” to bind to a predetermined nucleotide sequence, for example via engineering (altering one or more amino acids) of the recognition helix region of a naturally occurring zinc finger. Similarly, TALEs can be “engineered” to bind to a predetermined nucleotide sequence, for example by engineering of the amino acids involved in DNA binding (the repeat variable diresidue or RVD region). Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. No. 6,453,242; see also WO 98/53058; and U.S. Publication Nos. 2011/0301073, each of which is herein incorporated by reference.

A “selected” zinc finger protein, CRISPR or TALE is a protein not found in nature whose production results primarily from an empirical process such as phage display, interaction trap or hybrid selection.

In one embodiment, the polynucleotide encodes a zinc finger protein that binds to a gene encoding a Pirin-2 or a Pirin-4 polypeptide, resulting in reduced expression of the gene. In particular embodiments, the zinc finger protein binds to a regulatory region of a Pirin-2 or a Pirin-4. In other embodiments, the zinc finger protein binds to a messenger RNA encoding a Pirin-2 or a Pirin-4 polypeptide and prevents its translation. Methods of selecting sites for targeting by zinc finger proteins have been described, for example, in U.S. Pat. No. 6,453,242, and methods for using zinc finger proteins to inhibit the expression of genes in plants are described, for example, in US2003/0037355, each of which is herein incorporated by reference.

In another embodiment, the polynucleotide encoded a TALE protein that binds to a gene encoding a Pirin-2 or a Pirin-4 polypeptide, resulting in reduced expression of the gene. In particular embodiments, the TALE protein binds to a regulatory region of a Pirin-2 or a Pirin-4. In other embodiments, the TALE protein binds to a messenger RNA encoding a Pirin-2 or a Pirin-4 polypeptide and prevents its translation. Methods of selecting sites for targeting by TALE proteins have been described in e.g. Moscou M J, Bogdanove A J, 2009, A simple cipher governs DNA recognition by TAL effectors. Science 326:1501 and Morbitzer R, et al. 2010.

When the method involves increasing expression of a gene encoding a PIRIN4 polypeptide in the plant, the said PIRIN4 polypeptide is preferably selected from:

(a) a polypeptide consisting of the amino acid sequence shown as SEQ ID NO: 6;

(b) a polypeptide comprising the amino acid sequence shown as SEQ ID NO: 6;

(c) a polypeptide which is an ortholog in a plant species of the Arabidopsis thaliana PIRIN4 polypeptide,

which ortholog has at least 60% identity with the amino acid sequence shown as SEQ ID NO: 6; and which is capable of reducing lignin content in a plant.

In a preferred aspect, the PIRIN4 polypeptide, including orthologs thereof, has at least 60% identity, such as at least 70%, 75%, 80%, 85%, 90%, or 95%, with the amino acid sequence shown as SEQ ID NO: 6. However, the invention encompasses polypeptides carrying modifications like substitutions, small deletions, insertions or inversions, which polypeptides nevertheless have substantially the biological activities of a PIRIN4 polypeptide.

In a corresponding manner, the said gene encoding a PIRIN4 polypeptide is selected from:

(a) a nucleic acid molecule consisting of the nucleotide sequence shown as SEQ ID NO: 5;

(b) a nucleic acid molecule comprising the nucleotide sequence shown as SEQ ID NO: 5;

(c) a nucleic acid molecule which is capable of hybridizing, under stringent hybridization conditions, to a nucleotide sequence complementary to SEQ ID NO: 5; and

(d) a nucleic acid molecule which is an ortholog in a plant species of the Arabidopsis thaliana PIRIN4 gene, which ortholog has at least 65% identity with the nucleotide sequence shown as SEQ ID NO: 5; and which encodes a polypeptide capable of reducing lignin content in a plant. Preferably the stringent hybridization conditions in (c) are moderate stringency hybridization conditions, and more preferably high stringency hybridization conditions.

In a preferred aspect, the said gene encoding a PIRIN4 polypeptide, including orthologs thereof, has at least 65% identity, such as at least 70%, 75%, 80%, 85%, 90%, or 95%, with the nucleotide sequence shown as SEQ ID NO: 5. However, the invention encompasses polynucleotides carrying modifications like substitutions, small deletions, insertions or inversions, which nevertheless encode polypeptides having substantially the biological activity of the PIRIN4 polypeptide.

According to the invention, the plant is preferably:

(a) a hardwood selected from the group consisting of acacia, eucalyptus, hornbeam, beech, mahogany, walnut, oak, ash, willow, hickory, birch, chestnut, poplar, alder, maple, sycamore, ginkgo, palm trees, and sweet gum;

(b) a conifer selected from the group consisting of cypress, Douglas fir, fir, sequoia, hemlock, cedar, juniper, larch, pine, redwood, spruce, and yew;

(c) a fruit bearing plant selected from the group consisting of apple, plum, pear, banana, orange, kiwi, lemon, cherry, grapevine, papaya, peanut, and fig; or

(d) selected from the group consisting of cotton, bamboo, rubber plants, corn, rice, wheat, barley, Miscanthus, sorghum, ryegrass, sugarcane, and switchgrass.

In a more specific part of the invention the plant is acacia or eucalyptus.

In another specific part of the invention the plant is cotton, corn or rice.

In a further aspect, the invention provides a genetically modified plant produced by the method as defined above. Specifically, the plant is a genetically modified plant having modified expression of a gene encoding a PIRIN polypeptide; said plant comprising;

(a) a transgenic construct comprising a gene encoding a PIRIN2 polypeptide; or

(b) a transgenic construct decreasing the expression of the endogenous gene encoding a PIRIN4 in the plant;

wherein the amino acid sequence of the PIRIN2 polypeptide has at least 76% (preferably at least 80%, 85%, 90% or 95%) sequence identity with SEQ ID NO: 4, and wherein the amino acid sequence of the PIRIN4 polypeptide has at least 60% (preferably at least 70%, 75%, 80%, 85%, 90%, or 95%) sequence identity with SEQ ID NO: 6.

In yet another aspect the invention provides a method for producing a plant with enhanced lignin content by genetically manipulating the expression of a PIRIN gene, said method comprising:

(a) decreasing the expression of a gene encoding a PIRIN2 polypeptide in the plant; or

(b) increasing the expression of a gene encoding a PIRIN4 polypeptide in the plant;

wherein the amino acid sequence of the PIRIN2 polypeptide has at least 60% sequence identity to SEQ ID NO:4, and wherein the amino acid sequence of the PIRIN4 polypeptide has at least 60% sequence identity to SEQ ID NO: 6.

In yet another aspect the invention provides a method for producing a plant with an increased ratio between syringyl and guaiacyl lignin units (S/G ratio) by genetically manipulating the expression of a PIRIN gene, said method comprising:
(a) decreasing the expression of a gene encoding a PIRIN2 polypeptide in the plant; or
(b) increasing the expression of a gene encoding a PIRIN4 polypeptide in the plant;

wherein the amino acid sequence of the PIRIN2 polypeptide has at least 60% sequence identity to SEQ ID NO:4, and wherein the amino acid sequence of the PIRIN4 polypeptide has at least 60% sequence identity to SEQ ID NO: 6.

In yet another aspect the invention provides a method for producing a plant with enhanced caloric value by genetically manipulating the expression of a PIRIN gene, said method comprising:
(a) decreasing the expression of a gene encoding a PIRIN2 polypeptide in the plant; or
(b) increasing the expression of a gene encoding a PIRIN4 polypeptide in the plant;

wherein the amino acid sequence of the PIRIN2 polypeptide has at least 60% sequence identity to SEQ ID NO:4, and wherein the amino acid sequence of the PIRIN4 polypeptide has at least 60% sequence identity to SEQ ID NO: 6.

In yet another aspect the invention provides a genetically modified plant having modified expression of a gene encoding a PIRIN polypeptide; said plant comprising;
(a) a transgenic construct comprising a gene encoding a PIRIN4 polypeptide; or
(b) a transgenic construct decreasing the expression of the endogenous gene encoding a PIRIN2 in the plant;

wherein the amino acid sequence of the PIRIN2 polypeptide has at least 76% sequence identity to SEQ ID NO: 4, and wherein the amino acid sequence of the PIRIN4 polypeptide has at least 60% identity to SEQ ID NO: 6.

Genetically modified plants having an increased amount of lignin, as described herein above, can be used to manufacture fuels with improved caloric value, for instance, wood pellets which have a higher caloric value with respect to wood pellets manufactured from the non-modified plants.

In addition, genetically modified plants having an increased amount of lignin can be used to extract aromatic compounds, typically through processes such as pyrolysis or other processes that extract and/or depolymerize the lignin polymer, which aromatics can be used in the chemical industry.

Genetically modified plants having an increased S/G ratio, as described herein above, can be used to extract aromatic compounds, typically through processes such as pyrolysis or other processes that extract and/or depolymerize the lignin polymer, which aromatics can be used as building blocks in the chemical industry.

Genetically modified plants having an increased S/G ratio, as described herein above, can be used for their enhanced properties in delignification processes that rely on the cleavage of the ether bonds in the lignin polymer, such as the Kraft process.

By “plant” is intended whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen). Consequently, the invention also includes a plant cell, seed or progeny derived from the genetically modified plant as defined above.

Yet a further aspect of the invention is a method for the manufacture of wood pulp, said method comprising preparing a genetically modified woody plant as defined above; obtaining wood from the said woody plant; and converting the said wood into wood pulp.

Materials and Methods

Plant Material and Growth Conditions

Arabidopsis wild type Col-0 and Ler-0, pirin1 (SALK_006939), pirin2-1 (SM_3.15394), pirin2-2(SALK_079571), pirin3 (SAIL_1243), pirin4-1 (SALK_138671), pirin4-2 (SALK_125909), pirin4-3 (SALK_100855), pirin4-4 (GT_19099) were used. Soil grown plants were grown in growth chambers with short day conditions (8 hours light/16 hours darkness, 21° C./18° C., 70% relative humidity or 9 hours light/15 hours darkness, 22° C.) for at least eight weeks, followed by 16-h light/8-h dark, 22° C.

Cloning and Plant Transformation

2398 bp, 2308 bp, 527 bp, and 2427 bp fragment 5 prime of the AtPIRIN1, AtPIRIN2, AtPIRIN3, and AtPIRIN4 coding sequences, respectively, and full length cDNA of AtPIRIN2 and AtPIRIN4, were amplified and cloned into pDONR207 vector by BP Clonase II (Invitrogen) to make entry clones pENTR207pAtPIRIN1, pENTR207pAtPIRIN2, pENTR207pAtPIRIN3, pENTR207pAtPIRIN4, and pENTR207AtPIRIN2, pENTR207AtPIRIN4. Then pENTR207AtPIRIN2 and pENTR207AtPIRIN4 were recombined into pK2GW7 destination vector (Karimi et al. 2002). pENTR207pAtPIRI N1, pENTR207pAtPIRIN2, pENTR207pAtPIRIN3 and pENTR207pAtP/R/N4 were recombined into pMDC163 destination vector, respectively (Curtis and Grossniklaus 2003).

Histochemical GUS Staining

For histochemical GUS staining, in vitro grown seedlings or tissue pieces from soil grown plants were incubated for 90 min to 4 hrs or overnight at 37° C. in a solution containing 1 mM X-Gluc, 1 mM K₃Fe(CN)6, 1 mM K₄Fe(CN)6 and 0.1% Triton x-100 in 50 mM NaPO₄ buffer (pH7). Samples from soil grown plants were fixed in FAA (5% formaldehyde, 5% acetic acid, 50% ethanol), destained in 70% and 100% ethanol, rehydrated and embedded in 50% glycerol. Root samples were destained in 70% and 100% ethanol, rehydrated and embedded in 1M chloralhydrate in 33% glycerol. Light microscopic images were acquired using a Zeiss Axioplan II microscope equipped with Zeiss AxioCam CCD camera (Zeiss, Oberkochen, Germany).

Maule Staining

100 μm sections of 8 week-old Arabidopsis hypocotyls were made using Vibrotome (Leica). Sections were first rinsed with water then incubate in 0.5% KMnO₄ for 10 min followed by rinse with water and incubation in 10% HCl for 5 minutes. After a couple of times rinse with water, all sections were mounted in concentrated NH₄OH. Light microscopic images were acquired using a Zeiss Axioplan II microscope equipped with Zeiss AxioCam CCD camera (Zeiss, Oberkochen, Germany).

Pyrolysis-Gas Chromatography/Mass-Spectrometry (Py-GC/MS) Analysis

Freeze-dried hypocotyls were ball-milled (MM400, Retsch, Haan, Germany) at 30 Hz in stainless steel jars (1.5 mL) for 2 min with one ball (diameter 7 mm). A total of 35-50 pg of powder was then applied to the online pyrolizer (PY-2020iD and AS1020E, FrontierLabs, Japan) mounted on a GC/MS (7890A/5975C, Agilent Technologies AB Sweden, Kista, Sweden). Pyrolysis was conducted at 450° C. The pyrolysate was separated on a capillary column with a length of 30 m, diameter of 250 pm and film thickness of 25 pm (J&W DB-5, Agilent Technologies Sweden AB, Kista, Sweden). The GC oven temperature program started at 40° C., followed by an temperature ramp of 32° C./min to 100° C., 6° C./min to 118.75° C., 15° C./min to 250° C., and finally 32° C./min to 320° C. Total run time was 19 min and full-scan spectra were recorded in the range of 35-250 m/z. Data processing including peak detection, integration, normalization and identification was done as described in Gerber et al. (2012).

RT-qPCR Analysis:

Total RNA was prepared using the RNeasy® Plant Mini Kit (Qiagen) and treated with RQ1 RNase-Free Dnase (Promega) to remove traces of genomic DNA. Subsequent cDNA preparation was done using the iScript™ cDNA Synthesis Kit (Bio-Rad). Relative expression levels were determined with the LightCycler 480 Real-Time SYBR green PCR System (Roche). ACTIN (At3g18780), CDKA1 (At3g48750), UBQ10 (At4g05320) and SAND (At2g28390) were used as reference genes.

Phenolic Profiling

Plants for inflorescence stem material were grown and harvested similarly as described in Vanholme et al. (2012b). Stems were ground in a 2 mL Eppendorf tube with a 4 mm iron bead with a Retch ball mill. Metabolites were extracted by adding 1 mL methanol and exposing the samples to 70° C. under 1000 rpm shaking in a thermomixer for 15 min. After centrifugation, 800 μL of the liquid phase was lyophilized in a speedvac, 100 μL cyclohexane was added to dissolve the pellet followed by another 100 μL water. The tubes were vortexed and centrifuged at 14,000 rpm for 10 min. 15 μL of the lower water phase was injected on a Waters Acquity UPLC® system with a BEH C18 column (2.1×150 mm, 1.7 μM), coupled to a Synapt Q-Tof (Waters Corporation, Milford, Mass., USA). A gradient of two buffers was used: buffer A (99/1/0.1 H₂O/ACN/formic acid pH 3), buffer B (99/1/0.1 ACN/H₂O/formic acid pH3); 95% A for 0.1 min decreased to 50% A in 30 min (350 μL/min, column temperature 40° C.). The flow was coupled to the mass spectrometer equipped with an electrospray ionization source and lockspray interface for accurate mass measurements. The MS source parameters were capillary voltage, 1.5 kV; sampling cone, 40 V; extraction cone, 4 V; source temperature, 120° C.; desolvation temperature, 350° C.; cone gas flow, 50 L/h; and desolvation gas flow, 550 L/h. The collision energy for the trap and transfer cells was 6 V and 4 V, respectively. For data acquisition, the dynamic range enhancement mode was activated. Full-scan data were recorded in negative centroid V-mode; the mass range between m/z 100 and 1,000, with a scan speed of 0.2 s/scan, with MassLynx software (Waters). Leu-enkephalin (400 μg/μL solubilized in water/ACN 1:1 (vol:vol), with 0.1% formic acid) was used for the lock mass calibration, with scanning every 10 s with a scan time of 0.5 s. The values from three scans were averaged. The resulting chromatograms were integrated and aligned via MassLynx® software.

CWR, Acetylbromide and Thioacidolysis and Saccharification.

Cell wall experiments were performed as in Van Acker et al., 2013 except for that cellulose amount was not determined. Instead, cellulose to glucose was set to 21% conversion for WT at 48 h and all other data was normalized accordingly.

Tandem Affinity Purification (TAP)

TAP was performed as in Bassard et al., 2012. Cloning of transgenes encoding tag fusions under control of the cauliflower mosaic virus 35S promoter and transformation of Arabidopsis thaliana cell suspension cultures were performed as previously described in Van Leene et al., 2007. To scale up production, cells were subcultured in 50 mL of fresh medium without antibiotics at a 1:10 dilution and grown under standard conditions. After 1 week, cells were either harvested (non-=induced condition) or washed three times with 200 mL of adapted MSMO medium (4.43 g/L MSMO [Sigma-Aldrich], 30 g/L Suc, and 520 mg/L KH₂PO₄, pH 5.7) without hormones (induced condition). Washed cells were subsequently transferred to a one-liter shaker flask, diluted in 400 mL of the same medium used for washing, and incubated for 40 h at standard conditions. TAP of protein complexes was performed using the GS tag (Burckstummer et al., 2006) with the following protocol modifications. For all protein extractions prior to the affinity purification steps, the detergent Nonidet P-40 was replaced by digitonin (high purity; Calbiochem, Merck). Crude protein extracts were prepared in extraction buffer without detergent. After the mixing step, digitonin was added to a final concentration of 1% (w/v) and extracts were incubated for 1 h at 4° C. under gentle rotation. A soluble protein fraction was obtained by centrifugation at 36,900 g for two times 20 min at 4° C. In all further steps, the detergent 0.1% (v/v) Nonidet P-40 was replaced by 0.2% (w/v) digitonin. Protein precipitation and separation were done according to Van Leene et al. (2008). For the protocols of proteolysis and peptide isolation, acquisition of mass spectra by a 4800 Proteomics Analyzer (Applied Biosystems), and mass spectrometry-based protein homology identification based on The Arabidopsis Information Resource genomic database, we refer to Van Leene et al., 2010. Experimental background proteins were subtracted based on ˜40 TAP experiments on wild-type cultures and cultures expressing TAP-tagged mock proteins p-glucuronidase, RFP, and GFP.

### EXAMPLES

### Example 1

### The Expression of PIRIN2 and PIRIN4 Genes is Correlated with Genes Involved in Lignin Biosynthesis

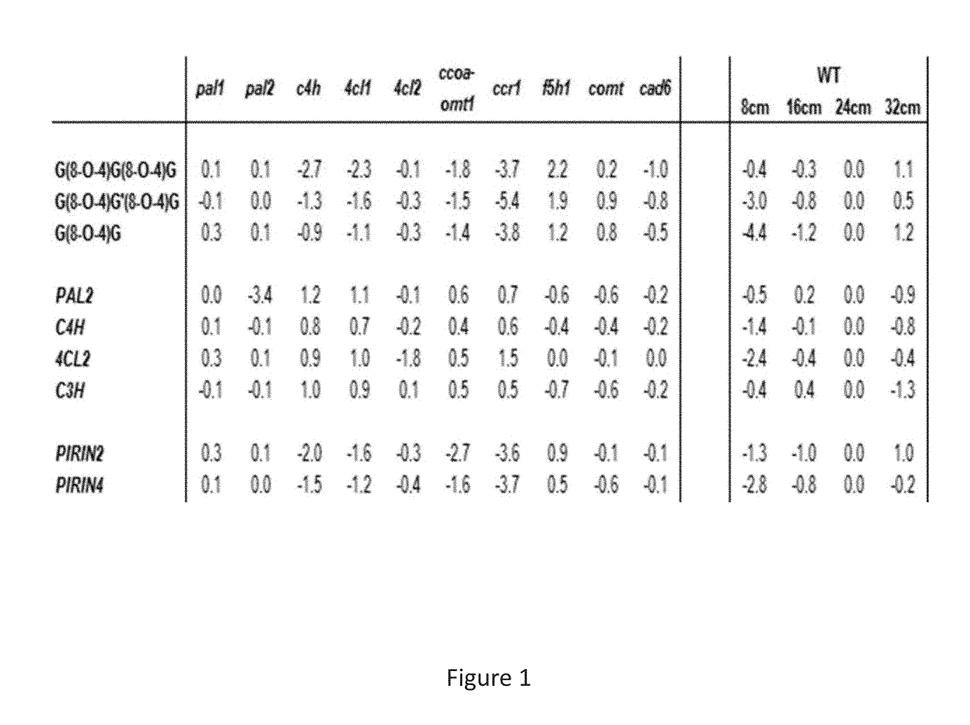

Re-evaluation of the data in Vanholme et al. (2012b) showed that the expression levels of two PIRIN genes was negatively correlated with the expression levels of genes involved in lignin biosynthesis in various mutants, but positively correlated with the expression levels of those genes in wild-type plants (FIG. 1). Interestingly, the transcript abundance of both PIRIN2 and PIRIN4 appeared to be positively correlated with the amount of G-type oligolignols (FIG. 1).

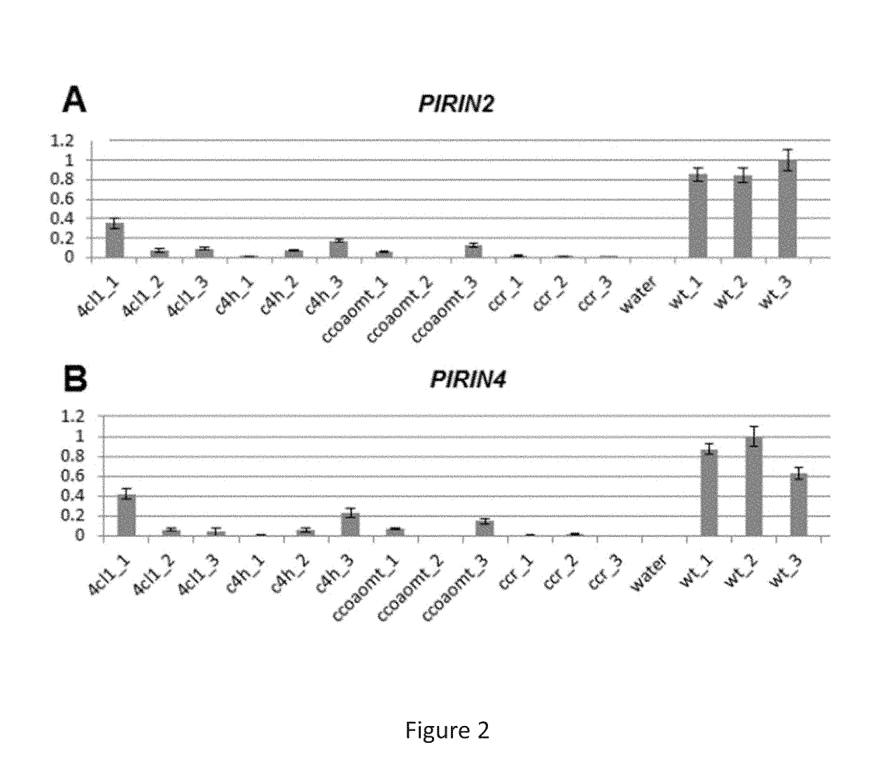

RT-qPCR was performed to provide further evidence for a reduced expression of PIRIN2 and PIRIN4 in c4h, 4c11, ccoaomtl and ccrl mutants. Therefore, the expression of PIRIN2 and 4 was measured in three biological replicates (and three technical replicates for each biological) of each mutant line and WT (FIG. 2). This RT-qPCR result confirmed the expression results as obtained by microarrays.

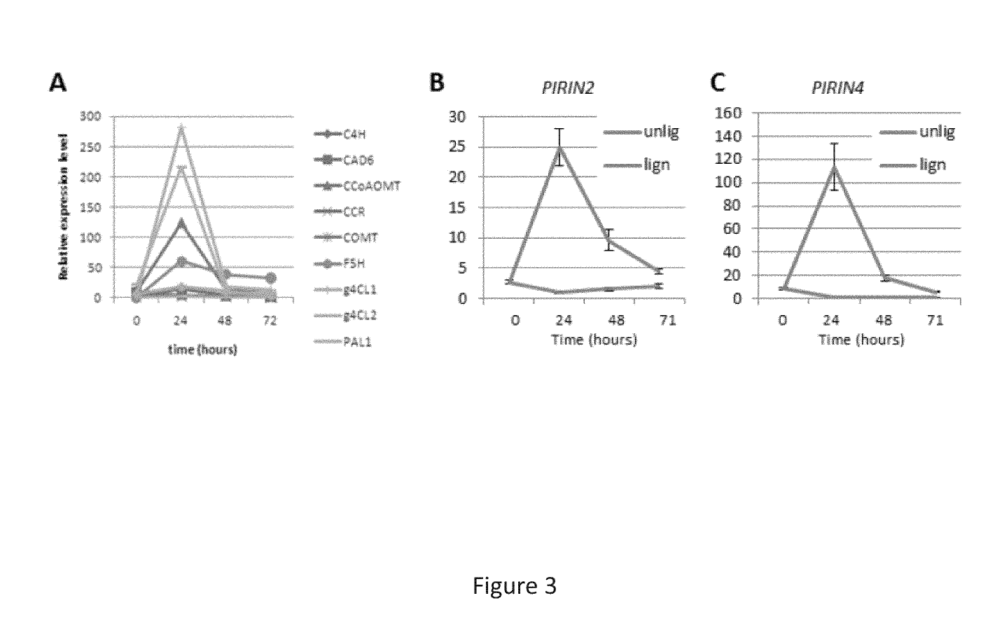

Further, the expression of PIRIN2 and PIRIN4 was investigated in by RT-qPCR in lignifying cell cultures, FIG. 3. It appeared that both PIRIN2 and PIRIN4 were up-regulated in cell cultures that were induced for lignification, whereas no up-regulation was detected when no induction was applied. In addition, both PIRIN2 and PIRIN4 have a similar expression profile to genes involved in lignification (4CL1, PALI, CCoAOMTI, COMT): These genes have a strong increase in expression after 24 h of induction, whereas the expression drops again after 48 and 71 hours. Suggesting again a role for PIRIN2 and PIRIN4 in lignification.

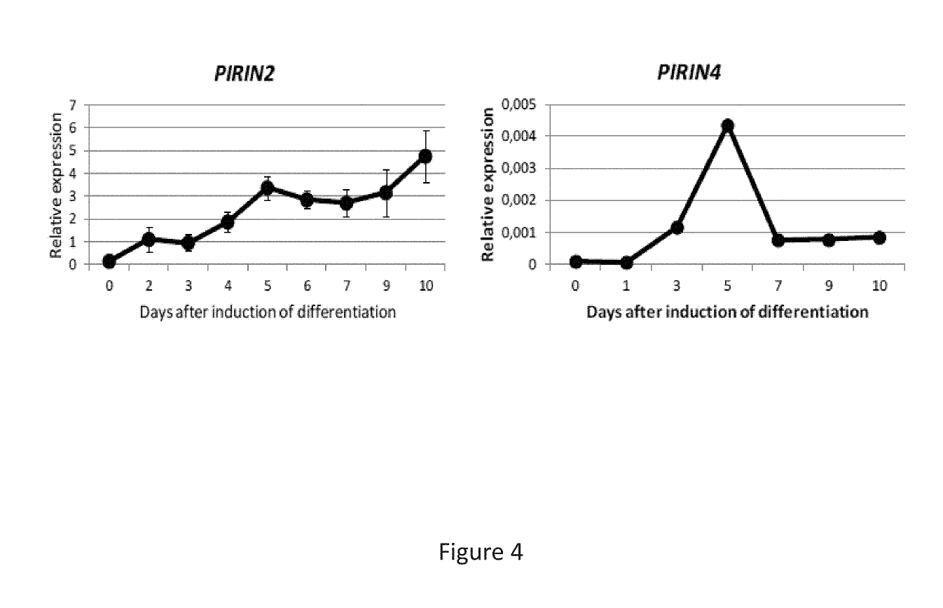

Further, RT-qPCR analysis was performed to validate PIRIN2 and PIRIN4 expression in lignification, using Arabidopsis Tracheary Element (TE) cell cultures (FIG. 4). Interestingly, both PIRIN2 and PIRIN4 have an expression peak at the fifth day when the maximum number of TE cells differentiated. However PIRIN2 expression stayed at a high level after 5 days while PIRIN4 dropped down. These data suggested diverse function of PIRIN2 and PIRIN4 in lignification.

The expression of PIRIN2 was investigated by RT-PCR in planta. It was shown that PIRIN2 is expressed ubiquitously throughout the plant.

Arabidopsis transgenic plants expressing p-glucuronidase (GUS) driven by each of the PIRIN gene promoters, were prepared. Samples from seedling roots, cotyledon, inflorescence stems, flowers, and hypocotyls were analyzed by histochemical GUS staining. PIRIN2 and PIRIN4 were shown to be expressed mainly in xylem. PIRIN2 is expressed mainly in the xylem parenchyma cells, while PIRIN4 is expressed both in developing xylem vessels and parenchyma cells.

### Example 2

### Identification of AtPIRINs Knockout Mutants and Overexpressing Transgenic Plants

To investigate the function of the Arabidopsis PIRIN proteins, several T-DNA insertion lines were screened for each of the PIRIN genes from the NASC (Nottingham Arabidopsis Stock Centre) collection (http://Arabidopsis.info/). One PIRIN1 homozygous knockout line (SALK_006939) was identified and named pirin1. Two PIRIN2 homozygous knockout lines, SM_3.15394 and SALK_079571 were identified and named pirin2-1 and pirin2-2, respectively. One PIRIN3 homozygous knockout line (SAIL_1243) was identified and named pirin3. Five PIRIN4 homozygous knockout lines were identified, including SALK_138671, SALK_125909, SALK_100855, GT19099 and GT25586, and named pirin4-1, pirin4-2, pirin4-3, pirin4-4 and pirin4-5, respectively. No full length according transcripts could be amplified by RT-PCR from each of the lines, pirin1, pirin2-1, pirin2-2, pirin3, and pirin4-4.

Further, two PIRIN2 overexpressing transgenic lines, PIRIN2OE6 and PIRIN2OE13, driven by cauliflower mosaic virus 35S promoter, were generated. Overexpression level of PIRIN2 was verified by RT-qPCR.

### Example 3

### Phenotype of Pirin2 Mutants and PIRIN2 Overexpression (PIRIN2 OE) Line

For phenotypic characterization, mutant and overexpression lines were grown alongside WT. After eight weeks of short day conditions that allowed development of a rosette but suppresses inflorescence stem development, plants were moved to long day conditions. No obvious effect could be observed on the development of the inflorescence of the pirin2 mutants and the PIRIN2 OE6 line. In addition, the final stem height was measured and no statistical differences were measured.

### Example 4

### Phenolic Profiling of Pirin2, Pirin4 and PIRIN2 OE

Because expression data suggested a role for PIRIN2 and PIRIN4 in lignin biosynthesis, a shift in the phenolic precursors of lignin (the oligolignols) was anticipated in the respective mutants and overexpression lines. The methanol-soluble phenolics of pirin mutants and overexpression lines were subjected to a detailed study via UHPLC-MS, a method referred to as phenolic profiling found in Vanholme et al., 2012a. First, the phenolics in hypocotyls of pirin2 mutants were investigated. A targeted analysis of the oligolignols in three pools of three hypocotyls each showed that S-unit containing oligolignols were increased in pirin2-2 mutants as compared to WT.

Further, methanol-soluble phenolics of inflorescence stems were investigated. Inflorescence stems were harvested at a height of about 24 cm. No significant differences between the phenolic composition of WT and the pirin2-2 mutants were detected. However, a further experiment was performed where pirin2-2 stems at different developmental stages were compared with WT. For this, stems of 30, 34 and 44 cm in height were used. Interestingly, targeted search for oligolignols did not result in any significant oligolignol at the 30 and 34 cm stages, but oligolignols were significant increased at the 44 cm stage (cut-of parameters: sign t-test (>0.01) and fold-change>2 or <0.5). The absence of phenolic phenotype in young stadia confirmed the data of the previous experiment with stems of 24 cm in height. Furthermore, the fact that a phenolic phenotype is only observed in a later developmental stage, hints that PIRIN2 has a role at this stage.

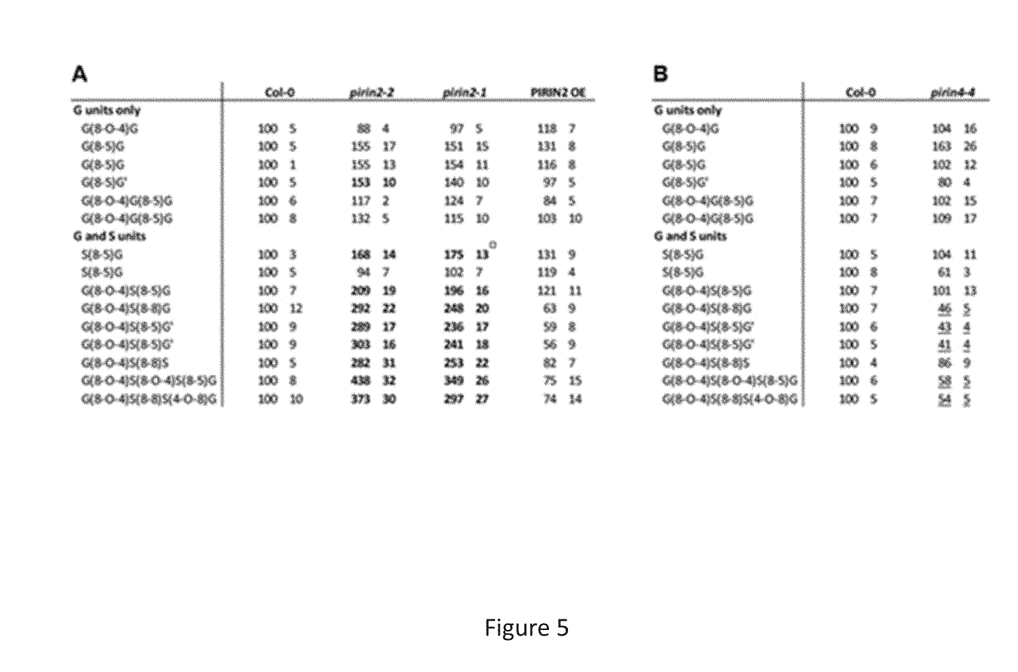

The methanol-soluble phenolics of pirin2 and pirin4 mutants and PIRIN2 OE6 were determined (FIG. 5). For pirin2-1, pirin2-2, and PIRIN2 OE, which are in the Col-0 background, stems of about 48 cm in height were used, whereas for pirin4 mutants which are in the Ler background, stems of about 28 cm were used. A significant increase in S-unit containing oligolignols was observed in both pirin2 mutants. A tendency for accumulation of oligolignols that are composed of G-units only is observed, but this tendency was in general not significant. Because acetyl bromide lignin is positively correlated (R²=0.80) with the amount of oligolignols, Vanholme et al., 2012b. The results here support the findings of 1) an increased S/G ratio in the lignin of the pirin2 mutants, and 2) an increase in acetylbromide in the pirin2 mutants, as compared to WT. No obvious shifts in phenolics of PIRIN2 OE6 lines were observed. In addition, several S-unit containing oligolignols were significantly reduced in pirin4 mutants, whereas oligolignols that contained G-units only were not significantly changed as compared to WT. These observations hints at a decreased S/G ratio in the lignin of the pirin4 mutants.

### Example 5

### Lignin Analysis by Thioacidolysis

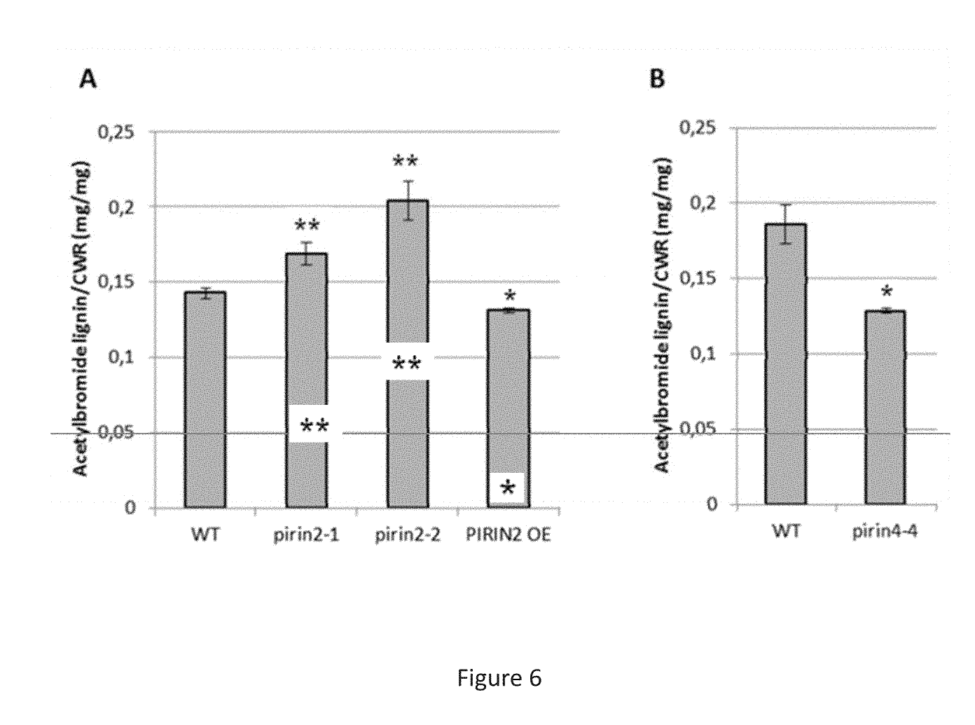

To further investigate the consequences of altering the expression of PIRINs, lignin analysis was performed for each of the above-mentioned lines. First, the fraction of the dry weight that is made up by extractives-free cell wall residue (CWR) was determined. However, no significant differences in CWR were measured for the pirin2 and pirin4 mutants and neither for the PIRIN2 OE6 line, as compared to WT. Next, the fraction of acetylbromide released lignin from the CWR was determined. For pirin2-1 and pirin2-2 mutants, the lignin amount increased by 18% and 43%, respectively, as compared to WT (FIG. 6). In addition, thioacidolysis showed that the lignin of the pirin2-2 mutant was enriched in S units.

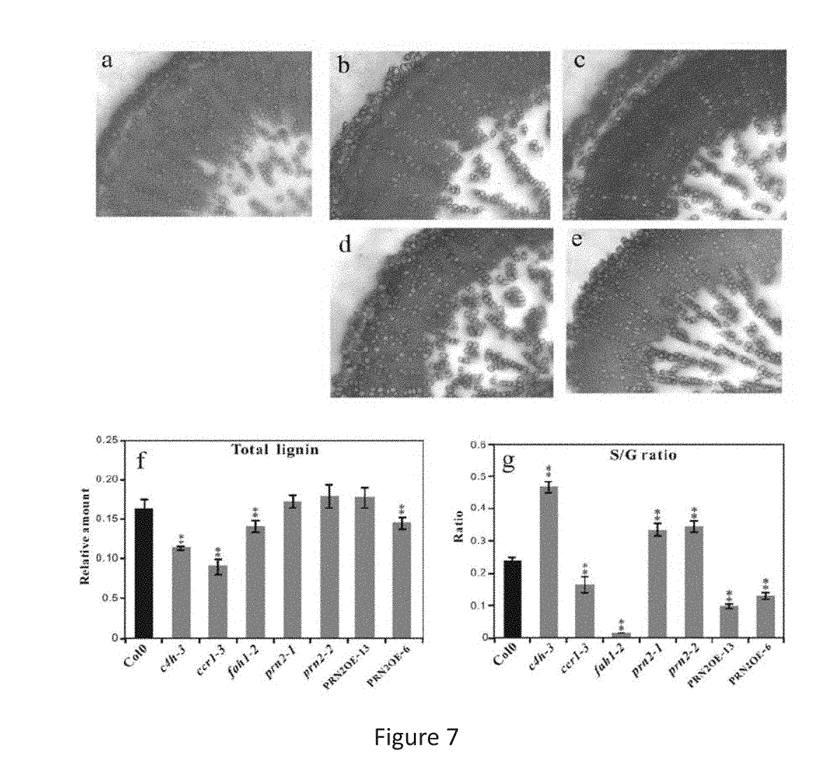

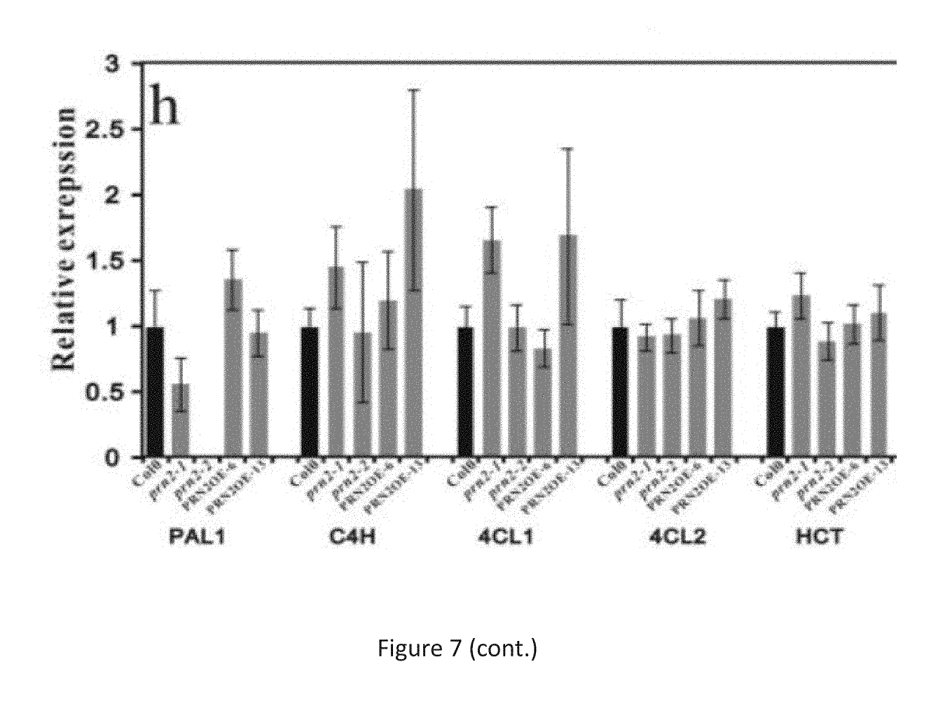

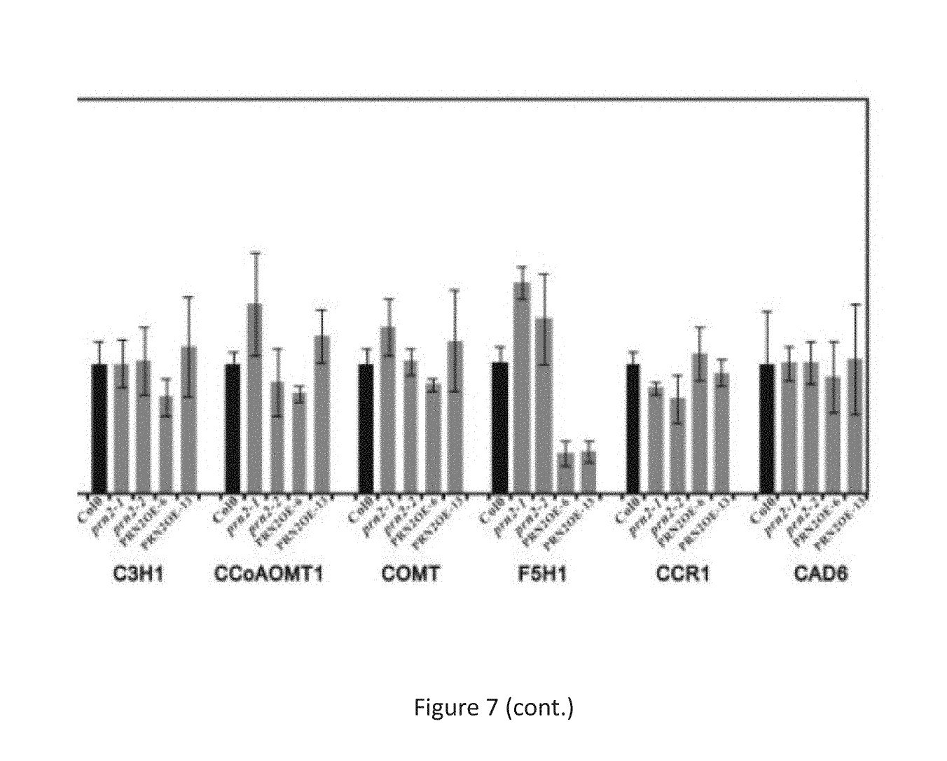

In the PIRIN2 OE6 line, acetylbromide lignin decreased by 8% or as compared to WT, FIG. 7. In the pirin4-4 line, acetylbromide lignin decreased by 31% as compared to WT (FIG. 6).

### Example 6

### Lignin Analysis by Pyrolysis-Gas Chromatography/Mass Spectrometry (Py-GC/MS)

Py-GC/MS analysis, see Materials and Methods, was performed to investigate cell wall chemistry of the pirin mutants. The analysis yielded chemical fingerprints consisting of 127 pyrolytic degradation products from the cell wall carbohydrates (cellulose and hemicellulose) and lignin (Gerber et al., 2012). Eight-week-old hypocotyls of single mutants including pirin1, pirin2-1, pirin2-2, pirin3, pirin4-1, pirin4-2, pirin4-3, and double mutants, pirin1 pirin2-1, pirin1 pirin2-2, pirin2-1 pirin3, pirin2-2 pirin3, pirin2-2 pirin4-1, pirin1 pirin4-1 were analyzed and compared to wild type. Interestingly, significant lignin composition changes compared to wild type were observed for the two pirin2 single mutants and double mutant crossed with pirin2 mutants.

Further analysis was carried out to confirm the lignification phenotype of the pirin knockout mutants and the OE lines. Maule staining, see Materials and Methods, was performed for eight-week-old hypocotyls, FIG. 7a-e. Compared to wild type two knockout mutants pirin2-1 and pirin2-2 showed more reddish staining, indicating that the secondary cell wall possessed more S-unit lignin, while more brownish staining in PIRIN2 overexpressing lines indicating more G-unit lignin containing cell wall. Py-GC/MS was performed to investigate cell wall chemistry of PIRIN2 overexpressing lines (FIGS. 7f and 7g). Two overexpressors showed lower S/G ratios compared to wild type which was the opposite phenotype to knockout mutants. Real-time qPCR was then performed to check transcriptional level of lignin biosynthetic genes (FIG. 7h). Interestingly, the S-unit biosynthesis gene F5H showed higher expression in the mutants while significant lower expression was observed in the overexpressors. This data indicated that lignin composition change resulted from changes in expression of some of the lignin biosynthesis genes.

### Example 7

### Saccharification

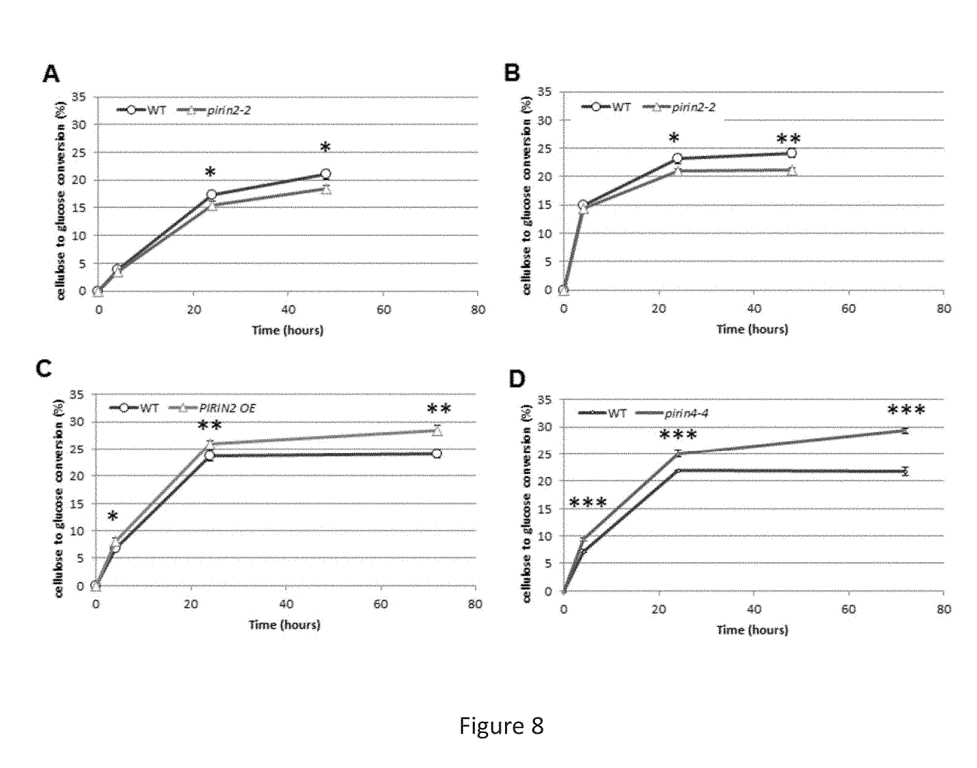

Since lignin is the major factor limiting saccharification of lignocellulosic biomass and since the lignin amount is altered in pirin2 and pirin4 mutants and in the PIRIN2 OE6 line, it was investigated whether these lines also had an altered saccharification yield. Therefore, CWR was treated with a mix of cellulases and cellobiase and the glucose released was measured over a period of two to three days, Van Acker et al., 2013. In agreement with the negative correlation of lignin amount and saccharification, the results showed a significant reduction of saccharification yield in pirin2 mutants (both with and without pretreatment) and a significant increase of saccharification yield in pirin4 mutants and PIRIN2 OE6 as compared to wild-type plants, FIG. 8.

### Example 8

### Tandem Affinity Purification (TAP)

To investigate possible interacting proteins of PIRIN2 and PIRIN4, both of them were used as baits in Tandem Affinity Purification (TAP) experiments, see Materials and Methods. Both PIRINs were once N- and once C-terminally tagged with the protein G-Streptavidin (GS) tag (Burckstummer et al., 2006), which resulted in four different TAP-constructs. These constructs were expressed in Arabidopsis cell suspension cultures (Van Leene et al., 2007). TAP-tagging was performed in cells grown under conditions that induced differentiation to treachery elements and secondary wall deposition, which also enhances expression of the lignin pathway. The 24 hours' time point after induction was used as these conditions had a relative high expression of both PIRIN2 and PIRIN4 (Oda et al., 2005). Using the C-terminally tagged PIRIN2 as bait, KAB1 one was co-purified in the two independent biological replicates. On the other hand both MPPBETA and Cytochrome bd ubiquinal oxidase were co-purified once. No interactors were co-purified when the N-terminal tag was used for PIRIN2. In the case of PIRIN4, a protein kinase was co-purified in all four independent replicates (N-terminal and C-terminal fusion, two replicates each). In addition, the N-terminal TAP of PIRIN resulted in an alpha/beta hydrolase protein, and PLDPT phospholipase that were found twice and LPD1 that was found in one of the replicates.

Interestingly, the expression of KAB1 was positively correlated with the expression of PIRIN2 in Arabidopsis mutants and over WT stem development. In addition, the expression of many other interactors was positively correlated with those of PIRIN2 or PIRIN4, over WT development. This positive correlation support the hypothesis that these genes are involved in the same biological process.

### REFERENCES

Agrawal, N. et al (2003) RNA Interference: Biology, Mechanism, and Applications. Microbiol Mol Biol Rev. 67(4): 657-685.
Bassard, et al. (2012) Protein-Protein and Protein-Membrane Associations in the Lignin Pathway. Plant Cell 24: 4465-4482
Baucher, M. et al. (2003) Lignin: Genetic engineering and impact on pulping. Critical Reviews in Biochemistry and Molecular Biology 38: 305-350.
Boerjan, W. et al. (2003) Lignin biosynthesis. Annual Review of Plant Biology 54: 519-546.
Burckstummer T. et al. (2006) An efficient tandem affinity purification procedure for interaction proteomics in mammalian cells. Nat. Methods 3: 1013-1019.
Chen, F. and R. A. Dixon (2007) Lignin modification improves fermentable sugar yields for biofuel production. Nature Biotechnology 25: 759-761.
Chiang, V. L. (2006) Monolignol biosynthesis and genetic engineering of lignin in trees, a review. Environmental Chemistry Letters 4: 143-146.
Curtis, M. D. and Grossniklaus, U. (2003) A gateway cloning vector set for high-throughput functional analysis of genes in planta. Plant Physiol, 133: 462-469
Dien, B. S. et al. (2011) Enhancing alfalfa conversion efficiencies for sugar recovery and ethanol production by altering lignin composition. Bioresour Technol. 102: 6479-86
Gerber, L. et al. (2012) Multivariate curve resolution provides a high-throughput data processing pipeline for pyrolysis-gas chromatography/mass spectrometry. J. Anal. Appl. Pyrolysis 95: 95-100.
Gomez, L. D. et al. (2008) Sustainable liquid biofuels from biomass: the writing's on the walls. New Phytologist 178: 473-485.
Halpin, C. (2004) Investigating and manipulating lignin biosynthesis in the postgenomic era. Advances in Botanical Research Incorporating Advances in Plant Pathology 41: 63-106.
Jinek et al (2012) A Programmable Dual-RNA—Guided DNA Endonuclease in Adaptive Bacterial Immunity Science 337, p. 816-821.
Karimi et al. 2002 Gateway vectors for Agrobacterium-mediated plant transformation. Trends Plant Sci 5 193-195.
Krysan, P. J. et al. (1999) T-DNA as an insertional mutagen in Arabidopsis. Plant Cell, 11: 2283-2290.
Li, X. et al. (2008) Improvement of biomass through lignin modification Plant Journal 54: 569-581.
Moscou M J, Bogdanove A J, (2009), A simple cipher governs DNA recognition by TAL effectors. Science 326:1501
Morbitzer R, et al. (2010) Regulation of selected genome loci using de novo-engineered transcription activator-like effector (TALE)-type transcription factors. Proc Natl Acad Sci USA 107:21617-21622.
Oda Y. et al. (2005) Regulation of secondary cell wall development by cortical microtubules during tracheary element differentiation in Arabidopsis cell suspensions. Plant Physiol. 137: 1027-1036.
Pesquet, E. et al. (2013) Non-cell autonomous postmortem lignification of tracheary elements in vascular plants. Plant Cell 25: 1314-1328
Pilate, G. et al. (2002) Field and pulping performance of transgenic trees with altered lignification. Nature Biotechnology 20: 607-612.
Studer, M. H. et al. (2011) Lignin content in natural Populus variants affects sugar release. Proc Natl Acad Sci U S A. 108: 6300-6305.
Van Acker, R. et al. (2103) Lignin biosynthesis perturbations affect secondary cell wall composition and saccharification yield in Arabidopsis thaliana. Biotechnology for Biofuels 6: 46.
Vanholme, R. et al. (2012a) Metabolic engineering of novel lignin in biomass crops. New Phytologist 196: 978-1000.
Vanholme, R. et al. (2012b) A systems biology view of responses to lignin biosynthesis perturbations in Arabidopsis. Plant Cell 24: 3506-3529.
Van Leene, J. et al. (2007) A tandem affinity purification-based technology platform to study the cell cycle interactome in Arabidopsis thaliana. Mol. Cell. Proteomics 6: 1226-1238.
Van Leene, J. et al. (2008) Boosting tandem affinity purification of plant protein complexes. Trends Plant Sci. 13: 517-520.
Van Leene, J. et al. (2010) Targeted interactomics reveals a complex core cell cycle machinery in Arabidopsis thaliana. Mol. Syst. Biol. 6: 397.

1. A method for producing a plant with reduced lignin content by genetically modifying expression of a PIRIN gene, said method comprising:
(a) increasing expression of a gene encoding a PIRIN2 polypeptide in the plant; or
(b) decreasing expression of a gene encoding a PIRIN4 polypeptide in the plant;
wherein the amino acid sequence of the PIRIN2 polypeptide has at least 60% sequence identity to SEQ ID NO: 4, and wherein the amino acid sequence of the PIRIN4 polypeptide has at least 60% sequence identity to SEQ ID NO: 6.

2. The method according to claim 1, wherein the plant has a decreased ratio between syringyl and guaiacyl lignin units (S/G ratio) as compared to a corresponding wild-type plant.

3. The method according to claim 1, wherein the plant has increased saccharification potential as compared to a corresponding wild-type plant.

4. The method according to claim 1 comprising increasing expression of a gene encoding a PIRIN2 polypeptide in the plant.

5. The method according to claim 4, wherein the plant is transformed with a transgene encoding the PIRIN2 polypeptide.

6. The method according to claim 5, wherein the transgene comprises a homologous or heterologous promoter operably linked to a DNA molecule encoding the PIRIN2 polypeptide.

7. The method according to claim 4, wherein the said PIRIN2 polypeptide is selected from the group consisting of:
(a) a polypeptide consisting of the amino acid sequence shown as SEQ ID NO: 4;
(b) a polypeptide comprising the amino acid sequence shown as SEQ ID NO: 4; and
(c) a polypeptide which is an ortholog in a plant species of the Arabidopsis thaliana PIRIN2 polypeptide, which ortholog has at least 60% identity with the amino acid sequence shown as SEQ ID NO: 4; and which is capable of reducing lignin content in a plant.

8. The method according to claim 7 wherein the PIRIN2 polypeptide has at least 80% identity with the amino acid sequence shown as SEQ ID NO: 4.

9. The method according to claim 4, wherein the said gene encoding a PIRIN2 polypeptide is selected from the group consisting of:
(a) a nucleic acid molecule consisting of the nucleotide sequence shown as SEQ ID NO: 3;
(b) a nucleic acid molecule comprising the nucleotide sequence shown as SEQ ID NO: 3;
(c) a nucleic acid molecule which is capable of hybridizing, under stringent hybridization conditions, to a nucleotide sequence complementary to SEQ ID NO: 3; and
(d) a nucleic acid molecule which is an ortholog in a plant species of the Arabidopsis thaliana PIRIN2 gene, which ortholog has at least 65% identity with the nucleotide sequence shown as SEQ ID NO: 3; and which encodes a polypeptide capable of reducing lignin content in a plant.

10. The method according to claim 9 wherein the gene encoding a PIRIN2 polypeptide has at least 80% identity with the nucleotide sequence shown as SEQ ID NO: 3.

11. The method according to claim 1, comprising decreasing expression of a gene encoding a PIRIN4 polypeptide in the plant.

12. The method according to claim 11, wherein expression of the gene encoding the PIRIN4 polypeptide in the plant is decreased by means of RNA interference or T-DNA insertion.

13. The method according to claim 11, wherein the said PIRIN4 polypeptide is selected from the group consisting of:
(a) a polypeptide consisting of the amino acid sequence shown as SEQ ID NO: 6;
(b) a polypeptide comprising the amino acid sequence shown as SEQ ID NO: 6; and
(c) a polypeptide which is an ortholog in a plant species of the Arabidopsis thaliana PIRIN4 polypeptide, which ortholog has at least 60% identity with the amino acid sequence shown as SEQ ID NO: 6; and which is capable of reducing lignin content in a plant.

14. The method according to claim 13 wherein the PIRIN4 polypeptide has at least 80% identity with the amino acid sequence shown as SEQ ID NO: 6.

15. The method according to claim 11, wherein the said gene encoding a PIRIN4 polypeptide is selected from the group consisting of:
(a) a nucleic acid molecule consisting of the nucleotide sequence shown as SEQ ID NO: 5;
(b) a nucleic acid molecule comprising the nucleotide sequence shown as SEQ ID NO: 5;
(c) a nucleic acid molecule which is capable of hybridizing, under stringent hybridization conditions, to a nucleotide sequence complementary to SEQ ID NO: 5; and
(d) a nucleic acid molecule which is an ortholog in a plant species of the Arabidopsis thaliana PIRIN4 gene, which ortholog has at least 65% identity with the nucleotide sequence shown as SEQ ID NO: 5; and which encodes a polypeptide capable of reducing lignin content in a plant.

16. The method according to claim 15 wherein the gene encoding a PIRIN4 polypeptide has at least 80% identity with the nucleotide sequence shown as SEQ ID NO: 5.

17. The method according to claim 1, wherein the plant is:
(a) a hardwood selected from the group consisting of acacia, eucalyptus, hornbeam, beech, mahogany, walnut, oak, ash, willow, hickory, birch, chestnut, poplar, alder, maple, sycamore, ginkgo, palm trees, and sweet gum;
(b) a conifer selected from the group consisting of cypress, Douglas fir, fir, sequoia, hemlock, cedar, juniper, larch, pine, redwood, spruce, and yew;
(c) a fruit bearing plant selected from the group consisting of apple, plum, pear, banana, orange, kiwi, lemon, cherry, grapevine, papaya, peanut, and fig; or
(d) selected from the group consisting of cotton, bamboo, rubber plants, corn, rice, wheat, barley, Miscanthus, sorghum, ryegrass, sugarcane, and switchgrass.

18. A genetically modified plant having modified expression of a gene encoding a PIRIN polypeptide; said plant comprising;
(a) a transgenic construct comprising a gene encoding a PIRIN2 polypeptide; or
(b) a transgenic construct capable of decreasing the expression of the endogenous gene encoding a PIRIN4 polypeptide in the plant;
wherein the amino acid sequence of the PIRIN2 polypeptide has at least 76% sequence identity to SEQ ID NO: 4, and wherein the amino acid sequence of the PIRIN4 polypeptide has at least 60% sequence identity to SEQ ID NO: 6.

19. A genetically modified plant produced by the method according to claim 1.

20. The genetically modified plant according to claim 18 which is:
(a) a hardwood selected from the group consisting of acacia, eucalyptus, hornbeam, beech, mahogany, walnut, oak, ash, willow, hickory, birch, chestnut, poplar, alder, maple, sycamore, ginkgo, palm trees, and sweet gum;
(b) a conifer selected from the group consisting of cypress, Douglas fir, fir, sequoia, hemlock, cedar, juniper, larch, pine, redwood, spruce, and yew;
(c) a fruit bearing plant selected from the group consisting of apple, plum, pear, banana, orange, kiwi, lemon, cherry, grapevine, papaya, peanut, and fig; or
(d) selected from the group consisting of cotton, bamboo, rubber plants, corn, rice, wheat, barley, Miscanthus, sorghum, ryegrass, sugarcane, and switchgrass.

21. A plant cell, seed or progeny derived from the genetically modified plant according to claim 18.


SEQUENCE LISTING (28 sequences)

SEQ ID NO: 1 - DNA, 864 bp, Arabidopsis thaliana
  CDS (1)..(864)
atgacgtatgagaacaatagtgtaccaagaattgtaatcaagaaagttctagcaaagcttgagaaggaaggtgaaggagctgtcgttagaaatggcatcacaaagattgaccagaagttattggacccgttcgtgttgctagttgaattttccttttcactctcagctggattcccagatcatcctcacagaggttttgaaagtgttacatacatgctacagggaggtatcattcacaaagatcctaaaggtcataaaggtacaattcaagccggagatgttcagtggatgacagcaggaagaggaatcattcattccgaatttccggaagaagaagtaaacaatggtttacagctttggatcaatctcccttccactgaaaaaatgactgaaccaaaatataaggaactatcgagtttagacattcctcgagcagaagaaaacggagttgaggtcaaagtcatagccggagattcaatgggaatcaaatctccagtctacacaagaacaccaacaatgttccttgactttaccctcaagccaggatctcaaacccaccaaaccgttccagaatcatggaccgctttcgcttacattatagaaggcgatgaaggtgttttcggttccttgaactcttccgcaatatcggcccaccatgttgttgtgtttgggccaggggatttagttagcgtgtggaacaagtcaacttcgaggtcattgaggtttttgttgattgcaggggaacctatcggcgagcctgtggttcagtgtggtccctttgtgatgaattcacaggcggagatcgatatggcttttgatgactatcagaatgctaagaacgggtttgaaatggccaagtgttag

SEQ ID NO: 2 - protein, 287 aa, Arabidopsis thaliana
MTYENNSVPRIVIKKVLAKLEKEGEGAVVRNGITKIDQKLLDPFVLLVEFSFSLSAGFPDHPHRGFESVTYMLQGGIIHKDPKGHKGTIQAGDVQWMTAGRGIIHSEFPEEEVNNGLQLWINLPSTEKMTEPKYKELSSLDIPRAEENGVEVKVIAGDSMGIKSPVYTRTPTMFLDFTLKPGSQTHQTVPESWTAFAYIIEGDEGVFGSLNSSAISAHHVVVFGPGDLVSVWNKSTSRSLRFLLIAGEPIGEPVVQCGPFVMNSQAEIDMAFDDYQNAKNGFEMAKC

SEQ ID NO: 3 - DNA, 966 bp, Arabidopsis thaliana
  CDS (1)..(966)
atgagagctgcaataaacagagcaaattcacttggaggtttgttttcatttaggtttatcagaaacatcaaatcaatgtcttcttctacttcccaagatttcgtatcaagacctgttatcaagaaagtcttcgctaagcttcagaaagaaggcgatggagccgtcgttagacgtggcatttccaggagtgagcagaagttgttagatcctttcttgatgctagatgaattctccgtttcgcctccagctggcttccctgatcatcctcacagagggtttgaaactgttacatacgtactagagggaggaatcactcaccaagacttcaaaggtcacaagggtacaatatatgctggtgatgttcagtggatgactgcaggaagaggaatcattcattctgaaatgcctgaagaagaagtcaacaaaggtttacaactttggatcaatctttcctccaacgagaaaatgatagaaccaaactaccaagaactgtcgcattcagacataccaaaagcagaacaaaacggtgttgaagtcaaagtcattgcaggagaatcaatgggaattcaatcacctgtttacacaagaacacctactatgttcctcgatttcactctccaaccaggagctcaaatccaccagaatgttccagaatcttggaacgcatttgcttacattctagagagcggtgaaggcggcggcgttttcagttcctcgaactcttcgcctattccagcacacagtgttgtggtgttcggaccagggaatgatggtgttagcgtgtggaacaagtcatcttcaaagcaattgaggtttgtgttgattgctggagaaccgattggtgaaccggtggttcaatacggtccgttcgtgatgaatactcaagctgagattgatatgaccattgaagattatcactatggtaagaatggcttcgaaatggccaagtattggaggtcacaatga

SEQ ID NO: 4 - protein, 321 aa, Arabidopsis thaliana
MRAAINRANSLGGLFSFRFIRNIKSMSSSTSQDFVSRPVIKKVFAKLQKEGDGAVVRRGISRSEQKLLDPFLMLDEFSVSPPAGFPDHPHRGFETVTYVLEGGITHQDFKGHKGTIYAGDVQWMTAGRGIIHSEMPEEEVNKGLQLWINLSSNEKMIEPNYQELSHSDIPKAEQNGVEVKVIAGESMGIQSPVYTRTPTMFLDFTLQPGAQIHQNVPESWNAFAYILESGEGGGVFSSSNSSPIPAHSVVVFGPGNDGVSVWNKSSSKQLRFVLIAGEPIGEPVVQYGPFVMNTQAEIDMTIEDYHYGKNGFEMAKYWRSQ

SEQ ID NO: 5 - DNA, 933 bp, Arabidopsis thaliana
  CDS (1)..(933)
atgccaatctcagagaaatcttctgctactaatacgaggttagttgttaagaagcttttcgccagacagctacatgaaggatttggtgctgttgtaagaagaagcatcggaaggtttgagttcagatacttcgacccatttcttgtcctggatgaattctcagtttcagctcctgcaggattcccagatcatccacacagaggatttgagacagtcacttacatgttagagggagagattttgcatgaagactgtgaaggacacaaaggagtaataagagaaggaggattacaatggatgactgcaggaaaaggcattgttcattctgaaatgccttcttctaattccaatggcattactcacaacaagggcttgcagctttggatcaatctctcctcaagacaaaaactagtggagccgagttatcaagagatagagagtaaagacatagcagaaacagagaaagatggtgtgagagttagagtcatagcaggagaatggaatggagttaagtcaaagatctgcacaagaacgccaacaatgtatttagacttcactctctcgccgggttctcgaatctctcaacccattccacttcactggaacgcgtttgtctatgttcttcaaggccatggtcactttggggattccaagttacaacattcagccgctgctgcgcaccacttgctggttctaggcctaggtggtgacatgttagaggcttggaacggttctgattccggtttgcctcttcggtttatccttgtggctggtgagcctataggtgaaccaatggttcagtttggtccttttgttatgaacacacaagaagagattgatgagaccattgacgactttgagaattttagaaatgggtttgaaaaagctagacattggaagtctcaagctgcttctgccttgggactgttctga

SEQ ID NO: 6 - protein, 310 aa, Arabidopsis thaliana
MPISEKSSATNTRLVVKKLFARQLHEGFGAVVRRSIGRFEFRYFDPFLVLDEFSVSAPAGFPDHPHRGFETVTYMLEGEILHEDCEGHKGVIREGGLQWMTAGKGIVHSEMPSSNSNGITHNKGLQLWINLSSRQKLVEPSYQEIESKDIAETEKDGVRVRVIAGEWNGVKSKICTRTPTMYLDFTLSPGSRISQPIPLHWNAFVYVLQGHGHFGDSKLQHSAAAAHHLLVLGLGGDMLEAWNGSDSGLPLRFILVAGEPIGEPMVQFGPFVMNTQEEIDETIDDFENFRNGFEKARHWKSQAASALGLF

SEQ ID NO: 7 - DNA, 978 bp, Populus trichocarpa
  CDS (1)..(978)
atgagagcttctatatatcgaaactttcttttcagattacatccaacaactactgccaaggctagtaagattaccaaccataccaggaatatcatgtctgaatcagatcaatccactggtttcagcacacccagattggtgaccaagaaggtcctggccaagctccaacatgaaggtgagggtgctgtcgttaggagaagcattggaaggagtgagctgaagttcttggatccttttctcatgttggatgatttttcagtgtccccccctgctggatttcctgatcatcctcacagaggttttgagactgttacatacatgctgcagggaggcatcactcatcaggattttgcaggacacaagggtacaatccataccggagatgtgcagtggatgacggcaggaagaggtatcatccactcagaaatgcctgctggagaaggtgagcagaagggtctgcagctgtggataaatctatcttcccaggacaaaatgattgaaccagggtatcaagaacttttaagcgatgacataaaaacagcagagaaagatggggttgaagtgagaattatagcagggaaatcaatgggagtgcagtctccagtttataccaggacaccaacaatgtatctggatttcactctgaaacctagagctcaaatgaatcaaagtattccagaatcatggaatgggtttgtgtatattattgaaggtgaaggggtctttggcaagccagcttcttcgcctgtaactgctcatcatgtcctggttttaggtcctggagatggtttaagtgtgtggaacaggtcttctaagccactgcgatttgtgcttattgccggacagccgattaacgagcctgtggttcagtatggaccttttgtgatgaacacacaggcagaaattgatcagaccattgaggactaccactatagcaaaaatgggtttgaaatggccaagtattggagatcccaatga

SEQ ID NO: 8 - protein, 325 aa, Populus trichocarpa
MRASIYRNFLFRLHPTTTAKASKITNHTRNIMSESDQSTGFSTPRLVTKKVLAKLQHEGEGAVVRRSIGRSELKFLDPFLMLDDFSVSPPAGFPDHPHRGFETVTYMLQGGITHQDFAGHKGTIHTGDVQWMTAGRGIIHSEMPAGEGEQKGLQLWINLSSQDKMIEPGYQELLSDDIKTAEKDGVEVRIIAGKSMGVQSPVYTRTPTMYLDFTLKPRAQMNQSIPESWNGFVYIIEGEGVFGKPASSPVTAHHVLVLGPGDGLSVWNRSSKPLRFVLIAGQPINEPVVQYGPFVMNTQAEIDQTIEDYHYSKNGFEMAKYWRSQ

SEQ ID NO: 9 - DNA, 978 bp, Populus trichocarpa
  CDS (1)..(978)
atgagagcttctttttatcgaaattttctgaagagattatatccaccaactactaccaaggctagtaacagtatacaccgtatcagaaatatcatgtctgaatcagatcaatccattggtttcgacacacccagattggtgaccaagaagatcctggccaagctccaacatgaaggtgatggtgctgttgttagaagaggcattggaaggagtgaacagaagttcttggatccttttctcatgctggatgatttttcagtgtctcctcctgctggatttcctgatcatccgcacagaggttttgagactgttacatacatgctgcagggaagcatcactcatcaggactttgcagggcacaagggtacaatccataccggagatgtgcagtggatgacagcaggaagaggcatcatccactcagaaatgcctgccggagaaggtgctcaaaagggtctgcagctgtggataaatctatcttcccaagacaaaatgattgaacctaggtatcaagaacttttaagtgatgacataaaaagagcagagaaagacggggctgaagtgagaattatagcaggggaatcgatgggagtgcgatctccagtttataccagaacaccaacaatgtatttggatttcactctgaaacccagagctcaagtgcatcaaaatatcccagaatcatggaatggatttgtgtatgttattgaaggtgaaggggtcttcggcaagccaaattcttcatcctcaactgctcatcatgtcctggttttaggtcctggagatggtttgagtgtgtggaacagatcttctaagccattgcgatttgtgctgattgccggacagccaattaacgagcctgtggttcagtatggaccttttgtgatgaacacagaggcagaaattgatcaaaccattgaggactaccactatagcaaaaatgggtttgaaatggccaagtattggagatcccaatga

SEQ ID NO: 10 - protein, 325 aa, Populus trichocarpa
MRASFYRNFLKRLYPPTTTKASNSIHRIRNIMSESDQSIGFDTPRLVTKKILAKLQHEGDGAVVRRGIGRSEQKFLDPFLMLDDFSVSPPAGFPDHPHRGFETVTYMLQGSITHQDFAGHKGTIHTGDVQWMTAGRGIIHSEMPAGEGAQKGLQLWINLSSQDKMIEPRYQELLSDDIKRAEKDGAEVRIIAGESMGVRSPVYTRTPTMYLDFTLKPRAQVHQNIPESWNGFVYVIEGEGVFGKPNSSSSTAHHVLVLGPGDGLSVWNRSSKPLRFVLIAGQPINEPVVQYGPFVMNTEAEIDQTIEDYHYSKNGFEMAKYWRSQ

SEQ ID NO: 11 - DNA, 1005 bp, Eucalyptus grandis
  CDS (1)..(1005)
atgagagcgaggtgtgatgggcggctactgctgatggtcctgtttctcggcttcacccacaggcttcttccactgttccatcctcgcctccctagcccctccccttccctcgtcaggagcgccatgtcccagcccgatttcagcaccccccgccccgtggtcaagcgggtcctggccaagcagcagcacgagggccagggcgccgtcgtccgcagaagcattggcaggaatgaattgaggagcttggatccctttctcatgctcgatgagttctcagtgtctcccccggctggatttcctgaccatcctcacagagggttcgaaactgtcacttacatgctccagggtgccttcacccatcaagatttcgctgggaacaggggaaccatacgaaccggcgatgtgcagtggatgacggcaggaagaggaatcatccactcggagatgccggcgggagaaggagtgaacaaggggctccagctctggataaatctctcctccagagacaaaatgattgagccacggtatcaggaattgcttagtgaggacataggcacagcagaggatgatggggtcgaagtccgggtgatcgcaggagaagcgatgggagtgaggtctccggtctacacaagaactccaactatgtaccttgattttactctcaagccaggagcaagattgaaccaaagcattcacgagtcatggaccgcttttgtgtacgtcatagaaggagaaggtgtttttggctccgcggattcgactcctgtgccagcgcacaacttgctggttctaggtccaggggatggggtgagcgtgtggaacgagtcttcgaagccgttgaggttcgtgctgataggcgggcagccgctgaatgagccggtggcgcaacacgggccttttgtgatgaacacgcccgctcaaattgatcaagccattgaagactatcaatatggcaagaacgggtttgaaatggccaggtactggaggtctcgatccgtgggttga

SEQ ID NO: 12 - protein, 334 aa, Eucalyptus grandis
MRARCDGRLLLMVLFLGFTHRLLPLFHPRLPSPSPSLVRSAMSQPDFSTPRPVVKRVLAKQQHEGQGAVVRRSIGRNELRSLDPFLMLDEFSVSPPAGFPDHPHRGFETVTYMLQGAFTHQDFAGNRGTIRTGDVQWMTAGRGIIHSEMPAGEGVNKGLQLWINLSSRDKMIEPRYQELLSEDIGTAEDDGVEVRVIAGEAMGVRSPVYTRTPTMYLDFTLKPGARLNQSIHESWTAFVYVIEGEGVFGSADSTPVPAHNLLVLGPGDGVSVWNESSKPLRFVLIGGQPLNEPVAQHGPFVMNTPAQIDQAIEDYQYGKNGFEMARYWRSRSVG

SEQ ID NO: 13 - DNA, 891 bp, Eucalyptus grandis
  CDS (1)..(891)
atgacggagagagaagtctcttgcgagatcagcgagccgcggttggtggccaggaagttcctggccaggcctcaacacgagggcgtcggggccatcgtcaggagaagcattggccggtttgagctgagatatttcgaccctttccttgtcttggatgaattttcagtgactgctcctgctggatttccagaccatccgcacagaggattcgagaccgtcacctacatgttacagggatccgtgacacatgaagatttcgaaggacacaaagggacgataggacccggtgatctacaatggatgaccgcggggagggggattgtccattccgagatgcctgcaagtcaaggaactcaaaaagggttacagctctgggtcaacctctcctccaaacacaaaatgactgaaccgaagtaccaagaaatatcaggcaaagacattgcagaagctgcagtagatgggatcaaagtgaaagtcatagcaggagaggctttaggagccaaatcccctgtctacacaagaacacccaccatgtacttggacttcaccctcaagccaggggctcacctgcagcagccgatacccgcctcgtggaacgccttcatctacatcctcgagggcgagggggtcttcgggatctcgaaatcctcgcccgtgaccgcccaccacctcctcctcctcggccacggagacggggtcgaggtgtggaacaaatccgccaagcccttgaggttcatcttggtcggaggcgagccgttgggcgagcccgtggcacagctcgggccgttcgtgatgaacactcaagaagagattgacaggacgatatatgatttcgaaaactgtatcaatgggttcgagaaggcaaggcattggaggagatcggacttttag

SEQ ID NO: 14 - protein, 296 aa, Eucalyptus grandis
MTEREVSCEISEPRLVARKFLARPQHEGVGAIVRRSIGRFELRYFDPFLVLDEFSVTAPAGFPDHPHRGFETVTYMLQGSVTHEDFEGHKGTIGPGDLQWMTAGRGIVHSEMPASQGTQKGLQLWVNLSSKHKMTEPKYQEISGKDIAEAAVDGIKVKVIAGEALGAKSPVYTRTPTMYLDFTLKPGAHLQQPIPASWNAFIYILEGEGVFGISKSSPVTAHHLLLLGHGDGVEVWNKSAKPLRFILVGGEPLGEPVAQLGPFVMNTQEEIDRTIYDFENCINGFEKARHWRRSDF

SEQ ID NO: 15 - DNA, 891 bp, Eucalyptus grandis
  CDS (1)..(891)
atgacggagagagaagtttcttgcgagatcagcgagccgcggttggtggccaggaagttcctggccaggcctcaacacgagggcgtcggggccatcgtcaggagaagcattggccggtttgagctgagatatttcgaccctttccttgccttggatgaattttcagtgactgctcctgctggatttccagaccatccgcacagaggattcgagaccgtcacctacatgttacagggatccgtgacacatgaagatttcgaagggcacaaagggacgataggacccggtgatctacaatggatgaccgcagggagggggattgtccattccgagatgcctgcaagtcaaggaactcaaaaagggttacagctctgggtcaacctctcctccaaacacaaaatgactgaaccgaagtaccaagaaatatcaggcaaagacattgcagaagctgcagtagatgggatcaaagtgaaagtcatagcaggagaggctttaggagccaaatcccctgtctacacaagaacacccaccatgtacttggacttcaccctcaaaccaggggctcacctgcagcagccaatacccacctcgtggaacgccttcgtctacgtccttgagggcgagggcgtcttcgggatctcgaaatcctcgcccgtgaccgcccaccacctcctcctcctcggccacggagacggggtcgaggtgtggaacaaatccgccaagcccttgaggttcatcttggtcggaggtgagccgttgggcgagcccgtggcgcagctggggccgttcgtgatgaacactcaagaagagattgacaggacgatatatgatttcgagaactgtatcaatgggttcgagaaggcaaggcattggaggagatcggacttttag

SEQ ID NO: 16 - protein, 296 aa, Eucalyptus grandis
MTEREVSCEISEPRLVARKFLARPQHEGVGAIVRRSIGRFELRYFDPFLALDEFSVTAPAGFPDHPHRGFETVTYMLQGSVTHEDFEGHKGTIGPGDLQWMTAGRGIVHSEMPASQGTQKGLQLWVNLSSKHKMTEPKYQEISGKDIAEAAVDGIKVKVIAGEALGAKSPVYTRTPTMYLDFTLKPGAHLQQPIPTSWNAFVYVLEGEGVFGISKSSPVTAHHLLLLGHGDGVEVWNKSAKPLRFILVGGEPLGEPVAQLGPFVMNTQEEIDRTIYDFENCINGFEKARHWRRSDF

SEQ ID NO: 17 - DNA, 1128 bp, Zea mays
  CDS (1)..(1128)
atgtctgccgccactgcttgcatgcccctggtccttgatccgtctcctctccatggatcggcggcaatggcagatgccgttgagaggcggcagcccggtggcatggagaccaccaagaccggttccgagggaacgctcggccgcgtcgtcaggagccaagtgtttggcaacgaggacgacgaccgtgctgggcacggcgacggcgacgaggtgagcaagccgcgggcggtggtccacacactgacgtgcgagcgcaagccattgttcgagggcttcgcgctctggcggagcatcggcaggcctgagcttcccgaactggatccaatcttatcgttcgacgaattcgagttttctgctccagctggcttccttgaccatcctcacaggggatttgagaacgttacctatatgctcgagggcggcatcagttaccacgacttttcaggccacaagggaaccatcaacccaggagacgttcagtggctgacggcggggcgcggcgtcgtgcacgcggagatgcccgccgccggccagggagtacagaggggcatcaacatctggatcaacctctctgccgccgacaagatggtggagccgaggtaccaggacctggcgagccacgacatccccgccgccgcggcggacgccgctggcggcgtctccgtcaaggtcatcgcgggcgagtgcctcggcgcgcgctccccgctccggccccgcaccccggcgctgtgcctcgacgtcgccctgcggcccggcgcgcgcctgcgccagcctgtcccgcgcgggtggagcgcctgcgcgtacgtcatccacggcgaggccgccttcggcactgcctccggcgagggtaacggcgcctccacggctaccgcgcgcacgctcgtcgtgttcggtggcgacggcgacggcgtggagctgcggggcgatgcagcgggccagggcgcgagggtcatgctggtcgcagctcggtcgcacggcgaggcggtggcgcgggacggccccttcgtcatgaatacccgggaggaggtggaccaggcgcgggaggactaccgccaccgccgcaacggcttcgagatggccgacggctggacctccgatcacgcctccgcagctgaaaagcattcatag

SEQ ID NO: 18 - protein, 375 aa, Zea mays
MSAATACMPLVLDPSPLHGSAAMADAVERRQPGGMETTKTGSEGTLGRVVRSQVFGNEDDDRAGHGDGDEVSKPRAVVHTLTCERKPLFEGFALWRSIGRPELPELDPILSFDEFEFSAPAGFLDHPHRGFENVTYMLEGGISYHDFSGHKGTINPGDVQWLTAGRGVVHAEMPAAGQGVQRGINIWINLSAADKMVEPRYQDLASHDIPAAAADAAGGVSVKVIAGECLGARSPLRPRTPALCLDVALRPGARLRQPVPRGWSACAYVIHGEAAFGTASGEGNGASTATARTLVVFGGDGDGVELRGDAAGQGARVMLVAARSHGEAVARDGPFVMNTREEVDQAREDYRHRRNGFEMADGWTSDHASAAEKHS

SEQ ID NO: 19 - DNA, 1140 bp, Zea mays
  CDS (1)..(1140)
atgacgacgacgatgatgaggctgaggcagcacctgggcctggtctcctcccctagtatttatacctccgccatcgccaggttcggcaacaacacaccccatcgccgccgaacaagtacatcaactactactcatatcacccaaaagcagttcctgctcctcgtgatcctcctgctcctcctcctcctcctcctcttctccgtcgtcttcttgatccccaccgtctcgttgcccccagctcggtctcggaccatgtcgtcgtccgcctccgacgccgcgccgttcgagaagcccagggccgtggtcaagaaggtcctcgcggagtcgcagcccgaggggcagggcgccaccgtccgcaggagcatcggcaggcatgagctccgcaacctggaccccttcctcctgctcgacgagttcacagtctacaagcccgccgggtttcccgaccacccccacagaggattcgagaccgtcacctacatgctcgagggggcattcacccaccaggacttcgctgggcacaagggcaccatcaagacaggggacgtgcagtggatgacggcggggcgaggcatcgtgcactcggagatgccggcgggggacggcgtgcacaagggcctgcagctctggatcaacctctcctccaaggacaagatgatcgagccgcggtaccaggagctggagagcaaggacatcagccgcggggagagcgaggacggcggcgtggaggcccgcgtcattgccggggaggccctcggcgcggcgtcccccgtgtacacgcgcacccccaccatgtacgtggacttcaccatgcgtcccgggtctcgcctgcaccagcccgtcccggagggctggaacgccttcgtctacgtcgtcgacggcgagggcgtgttcgggcgggagacggccacggcgcactactgcctcgtcctcggccccggcgacggcgtcagcgtgtggaacaggtccaccaggccgctgaggttcgtcctcgtcgccgggcagccgctcggcgagcccgtggtgcagcacgggcccttcgtcatgaactcgcgcgcccagattcagaaggccatggaggactactactatggcaagaacggcttcgagagggccggacagtggagctcctccgcctga

SEQ ID NO: 20 - protein, 379 aa, Zea mays
MTTTMMRLRQHLGLVSSPSIYTSAIARFGNNTPHRRRTSTSTTTHITQKQFLLLVILLLLLLLLLFSVVFLIPTVSLPPARSRTMSSSASDAAPFEKPRAVVKKVLAESQPEGQGATVRRSIGRHELRNLDPFLLLDEFTVYKPAGFPDHPHRGFETVTYMLEGAFTHQDFAGHKGTIKTGDVQWMTAGRGIVHSEMPAGDGVHKGLQLWINLSSKDKMIEPRYQELESKDISRGESEDGGVEARVIAGEALGAASPVYTRTPTMYVDFTMRPGSRLHQPVPEGWNAFVYVVDGEGVFGRETATAHYCLVLGPGDGVSVWNRSTRPLRFVLVAGQPLGEPVVQHGPFVMNSRAQIQKAMEDYYYGKNGFERAGQWSSSA

SEQ ID NO: 21 - DNA, 891 bp, Zea mays
  CDS (1)..(891)
atggagaagccccggcaggtggcgcggaaattcctggcccggccgcagcacgagggcgccggcgccgtcgtccgccgcagcatcggcaggttcgagctgaggtacttcgacccgttccttgtcctcgacgagttcacagtttctgctccggccggcttccccgaccacccgcaccgaggcttcgagaccgtcacctacatgcttgagggcgccgtgacgcacgaggacttcgagggccaccgtggcaccatcaaggccggcgacgtgcagtggatgacggccggccgcggcatcgtgcactccgagatgcccgccggccccggcacgtccaggggcctgcagctctgggtcaacctctcctccgccaacaaaatggtcgagcccgggtaccaggagatccagagcaaggacatcgcgtgcacgtccgacggcgacggcggcgtcacggtgcgcgtgatcgccggccacgccatgggcgtgcgctcgccggtgcgcacgcgcacgccgaccatgtacctggacttcacggtgcgcccgcgcggcgcggtgcggcagcccgtgcgcgcgtcctggaacgccttcgcctacgtgctggagggcgagggcgtgttcggcgccgagcggtgcgcgcccgtcggcgcccaccacctgctgctgctcggccacggcgacggcctcgaggtctggaacaagttgcccgaccgcccgctccgcttcctgctcgtcgccggcgagcccatcggggagcccgtcgcgcagctcggccccttcgtcatgaacaccgaggaggagatcgacatgaccgtcaacgacttcgagtgctacgccaatgggttcgagaaggcccgccactggaagtcgcaggcgatgctggcgcttggcgtagagtag

SEQ ID NO: 22 - protein, 296 aa, Zea mays
MEKPRQVARKFLARPQHEGAGAVVRRSIGRFELRYFDPFLVLDEFTVSAPAGFPDHPHRGFETVTYMLEGAVTHEDFEGHRGTIKAGDVQWMTAGRGIVHSEMPAGPGTSRGLQLWVNLSSANKMVEPGYQEIQSKDIACTSDGDGGVTVRVIAGHAMGVRSPVRTRTPTMYLDFTVRPRGAVRQPVRASWNAFAYVLEGEGVFGAERCAPVGAHHLLLLGHGDGLEVWNKLPDRPLRFLLVAGEPIGEPVAQLGPFVMNTEEEIDMTVNDFECYANGFEKARHWKSQAMLALGVE

SEQ ID NO: 23 - DNA, 954 bp, Zea mays
  CDS (1)..(954)
atggccacggtctggacaaaagccaggctgggcttgtggctggatgcccgagcggctcggtctcggatcgtgtcgtcctccgcctccgacgccgcgccgttcgagaagcccagggccgtggtcaagaaggtcctcgcgcagtcgcagcccgagggtcagggtgccaccgtccgtaggagcatcggcaggcacgagctccgcaacctggacccgttcctccagctcgacgagttcacagtctccaagcccgccgggtttcccgaccacccgcacagaggattcgagaccgtcacccacatgtttgagggggccatcacccaccaggacttcgctggtcacaagggcaccatcaggacaggggacgtgcagtggatgacggcggggcgaggcatcgtgcactcggagatgccggctggggacggcgtgagcgagggcctgcagctctggatcaacctctcctccaaggacaagatgatcgagccgcgataccaggagctggagcgcaaggacatcagccgcgcggagaccgaggacggcgtggaggcccgcgtcatcgccggggaggccttcggcgtggcgtcccccgtgtacacgcgcactcccatcatgtacgtggacttcaccatgcgacctgggtcccgcctgcaccagcctgtcccggagggctggaacgccttcgtctacgtcgtcgacggcgagggcgtattcggacgggagacggccacggcacaccactgcctcgtcctcggctccggcgacggcgtcagcgtgtggaacaggtccgccaggccgttaaggttcgtcctcgccgccgggcagccgctcggtgagcccgtggtgcagcacggacccttcgtcatgaactcgcacgccgagatacagcaggccatggaagactactcctacggcaagaacggcttcgagagggccggccagtggagctcctccgcctga

SEQ ID NO: 24 - protein, 317 aa, Zea mays
MATVWTKARLGLWLDARAARSRIVSSSASDAAPFEKPRAVVKKVLAQSQPEGQGATVRRSIGRHELRNLDPFLQLDEFTVSKPAGFPDHPHRGFETVTHMFEGAITHQDFAGHKGTIRTGDVQWMTAGRGIVHSEMPAGDGVSEGLQLWINLSSKDKMIEPRYQELERKDISRAETEDGVEARVIAGEAFGVASPVYTRTPIMYVDFTMRPGSRLHQPVPEGWNAFVYVVDGEGVFGRETATAHHCLVLGSGDGVSVWNRSARPLRFVLAAGQPLGEPVVQHGPFVMNSHAEIQQAMEDYSYGKNGFERAGQWSSSA

SEQ ID NO: 25 - DNA, 972 bp, Zea mays
  CDS (1)..(972)
atgatcgagccgcggtaccaggagcttgagcgcaaggacatcagccgcggggagagcgagaacgtcgacatggaggcccgcgtcatcgccggggaggccctcggcgtggcgtcccccgtgtacacgctcacccccaccatgtatgtggacttcaccatgcgccccgggtctcgcctgcaccagtctgtcctggagggctggaacgccttcatctacgtcatcgacggcgatggcgtgttcgggcgggcgacggccacggcgcactactgcctcgtcctcggccccggcgacggcgtcagcgtgtggaacaggtccaccaggccgctgaggttcgtcctcgtcgccggtcagccgctgggcgtgcccgtggtgcagcacaggcccttcgtcatgaactcgcgcgccgagattcagaaggccatggaagactactattatggcaagaacggctttgagagggccggacagtggagctcctccgccggatcggcgaggcgggaagctgatggggatcagtcatcaaattttagcatctatttgtcatgcagtcctaaatggactgatggcagcattcctttggatgtactttctgataaactcacaaaaataagcaagatcgctgctagccagaccaagatttacatggtgcttcagcttgtcaatgggggcaaactgttcgatggtattacacacaatgttaccaaggaattaatgaagtatcactcgcagactcttggatatgtaattggtggcaataacatgagaagcgacgccaaccatctcacagaagggatcaatatcttaattgccacactggacaggcttttggaccatctgcaaaataccggtagtttcaaatacaaagagctaaagagtgtgaaaaccactaggatctatgatgaggagtttgctgatttgggatggggaagaggcgaaaatgagattgaggatgtgtttatcgttgttttgtag

SEQ ID NO: 26 - protein, 323 aa, Zea mays
MIEPRYQELERKDISRGESENVDMEARVIAGEALGVASPVYTLTPTMYVDFTMRPGSRLHQSVLEGWNAFIYVIDGDGVFGRATATAHYCLVLGPGDGVSVWNRSTRPLRFVLVAGQPLGVPVVQHRPFVMNSRAEIQKAMEDYYYGKNGFERAGQWSSSAGSARREADGDQSSNFSIYLSCSPKWTDGSIPLDVLSDKLTKISKIAASQTKIYMVLQLVNGGKLFDGITHNVTKELMKYHSQTLGYVIGGNNMRSDANHLTEGINILIATLDRLLDHLQNTGSFKYKELKSVKTTRIYDEEFADLGWGRGENEIEDVFIVVL

SEQ ID NO: 27 - DNA, 489 bp, Zea mays
  CDS (1)..(489)
atgctcgaggttcgttgcgtctgctcaactcaatgcttcgctcctcttctcttctgttcaacgcacgcgagctgttcgctcaaacgtccctatccttgtggttccggttactggctgctgcagggggcattcacccaccaggacttcgctgggcacaagggcaccatcaggacaggggacgtgcagtggatgacggcggggcgaggcatcgtgcactcggagatgccggcgggggacgacgtgcacaagggcctgcaactctggatcaacctctcctctaaatacaagatgatcgagccgcagtaccaagagctggagcgcaaggacatcagccgcggggagagcgagaacggcggcgtggaggccctcggtgtggcgacccccgtgtacacgcacccccaccatgtatgtggacttcaccatgcgccccgggtctcgcctgcaccagcccgtcccggagggctggaatgccttcgtctacgtcgttga

SEQ ID NO: 28 - protein, 162 aa, Zea mays
MLEVRCVCSTQCFAPLLFCSTHASCSLKRPYPCGSGYWLLQGAFTHQDFAGHKGTIRTGDVQWMTAGRGIVHSEMPAGDDVHKGLQLWINLSSKYKMIEPQYQELERKDISRGESENGGVEALGVATPVYTHPHHVCGLHHAPRVSPAPARPGGLECLRLRR